(19) United States
(12) Patent Application Publication
(10) Pub. No.: US 2018/0216113 A1
(43) Pub. Date: Aug. 2, 2018

(54) METHODS FOR REDUCING PROTEOTOXICITY

(71) Applicant: The Regents of the University of California, Oakland, CA (US)

(72) Inventors: Andrew Dillin, Berkeley, CA (US); Hyun-Eui Kim, Berkeley, CA (US)

(21) Appl. No.: 15/747,433

(22) PCT Filed: Aug. 18, 2016
(86) PCT No.: PCT/US16/47577

§ 371 Date: Jan. 24, 2018

Related U.S. Application Data
(60) Provisional application No. 62/207,831, filed on Aug. 20, 2015.

(51) Int. Cl. C12N 15/113; A61K 31/336; A61K 31/4458; A61K 31/198; A61K 31/205; A61P 43/00

(52) U.S. Cl. CPC C12N 15/1137; A61K 31/336; A61K 31/4458; A61K 31/198; A61K 31/205; C12N 2310/531; C12N 2310/3233; C12N 2310/3515; C12N 2310/11; C12N 2310/14; A61P 43/00

(57) ABSTRACT

The present disclosure provides methods of reducing protein misfolding and/or aggregation in a cell. The present disclosure provides methods of treating diseases and disorders associated with protein misfolding and/or aggregation.

### CROSS-REFERENCE

This application claims the benefit of U.S. Provisional Patent Application No. 62/207,831, filed Aug. 20, 2015, which application is incorporated herein by reference in its entirety.

### INTRODUCTION

Defects in mitochondrial metabolism have been increasingly linked with age-onset protein misfolding diseases such as Alzheimer's, Parkinson's, and Huntington's. In response to protein folding stress, compartment-specific unfolded protein responses (UPRs) within the endoplasmic reticulum, mitochondria, and cytosol work in parallel to ensure cellular protein homeostasis. While perturbation of individual compartments can make other compartments more susceptible to protein stress, the cellular conditions that trigger cross-communication between the individual UPRs remain poorly understood.

### SUMMARY

The present disclosure provides methods of reducing protein misfolding and/or aggregation in a cell. The present disclosure provides methods of treating diseases and disorders associated with protein misfolding and/or aggregation.

### BRIEF DESCRIPTION OF THE DRAWINGS

FIG. 1A-1C depict the effect of knockdown of mitochondrial HSP70 (hsp-6) on the cytosolic heat shock response.

FIG. 2A-2C depict microarray analysis of the effect of DVE-1 and HSF-1 on gene regulation of fat metabolism.

FIG. 3A-3C depict the effect of knockdown of mitochondrial HSP70 (hsp-6) on fat storage.

FIG. 4A-4C depict the effect of reducing fat synthesis and inhibiting carnitine palmitoyltransferase (CPT) on cytosolic response.

FIG. 5A-5E depict the effect of mtHSP70 knockdown on cytosolic protein homeostasis in poly-Q expressing C. elegans and human primary fibroblasts.

FIG. 6 depicts the role of mtHSP70 in signaling from the mitochondria to the cytosol (MCSR) via alternating fat metabolism.

FIG. 7A-7E depict the effect of hsp-6 RNAi on a MCSR that is independent of mitochondrial import.

FIG. 8A-8B depict electron microscopy images of C. elegans after hsp-6 RNAi.

FIG. 9A-9E depict triglyceride content after RNAi treatment.

FIG. 10A-10B depict western blotting analysis of mtHSP70 and cytosolic mtHSP70.protein levels after siRNA treatment or perhexiline treatment on human primary fibroblasts.

FIG. 11 depicts a mtHSP70 amino acid sequence (SEQ ID NO:1).

FIG. 12A-12B depict a mtHSP70 nucleotide sequence (SEQ ID NO:2).

FIG. 13A-13F depict the requirement for cardiolipin synthesis for MCSR induction, and depict the effect of inhibition of ceramide synthesis on MCSR induction.

FIG. 14A-14F depict the requirement for cardiolipin synthesis for MCSR induction, and depict the effect of inhibition of ceramide synthesis on MCSR induction.

FIG. 15A-15C provides a table showing lipidomic analysis of hsp-6 RNAi-ed worms (hsp-6 RNAi treated worms).

### DEFINITIONS

The term “protein aggregate” refers to an accumulation of two or more misfolded proteins.

As used herein, the terms “treatment,” “treating,” “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease. In some cases, “treating” refers to inhibiting conception.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc.

A “therapeutically effective amount” or “efficacious amount” refers to the amount of an agent that, when administered to a mammal, is sufficient to effect a treatment (e.g., treatment of a disease; contraception; etc.). The “therapeutically effective amount” will vary depending on the agent, the disease and its severity and the age, weight, etc., of the subject to be treated.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a CPT inhibitor” includes a plurality of such inhibitors and reference to “the siRNA” includes reference to one or more siRNAs and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides methods of reducing protein misfolding and/or aggregation in a cell. The present disclosure provides methods of treating diseases and disorders associated with protein misfolding and/or aggregation.

Methods of Reducing Protein Misfolding and/or Aggregation

A method of reducing protein aggregation and/or protein misfolding in a cell, the method comprising contacting the cell with an agent that modulates a mitochondrial to cytosolic stress response in the cell.

Suitable agents include carnitine palmitoyltransferase (CPT) inhibitors, and nucleic acid agents that reduce the level of mitochondrial heat shock protein-70 (mtHSP70).

In some cases, an active agent (a CPT inhibitor; a nucleic acid agent that reduces the level of a mtHSP70 gene product), when contacted with a cell, is effective to reduce the level of protein aggregation in the cell, or in an extracellular fluid, by at least 10%, at least 20%, at least 30%, at least 40%, at least 50%, at least 60%, at least 70%, at least 80%, or more than 80%, compared to the level of protein aggregation in the cell, or in the extracellular fluid in the absence of the active agent.

In some cases, an active agent (a CPT inhibitor; a nucleic acid agent that reduces the level of a mtHSP70 gene product), when contacted with a cell, is effective to reduce the level of protein aggregates in the cell, or in an extracellular fluid, by at least 10%, at least 20%, at least 30%, at least 40%, at least 50%, at least 60%, at least 70%, at least 80%, or more than 80%, compared to the level of protein aggregates in the cell, or in the extracellular fluid, in the absence of the active agent.

In some cases, the protein aggregates are aggregates of proteins containing polyglutamine tracts (e.g., Huntington protein); amyloid beta (Aβ) protein; serpin; transthyretin; valosin containing peptide; tau protein; α-synuclein; superoxide dismutase; PABPN1; prion proteins; TDP-43; and the like. For example, wild-type Huntington protein includes fewer than 35 consecutive glutamines, while disease-associated Huntington protein can have 40 consecutive glutamines, or more than 40 consecutive glutamines. Pathological polyglutamine expansion proteins (and their related disorders) may include, but are not limited to, Huntington protein (Huntington's disease), androgen receptor (AR; spinobulbar muscular atrophy), ATN1 (dentatorubropallidoluysian atrophy), ATXN1 (Spinocerebellar ataxia Type 1), ATXN2, (Spinocerebellar ataxia Type 2), ATXN3, (Spinocerebellar ataxia Type 3), CACNA1A (Spinocerebellar ataxia Type 6), ATXN7 (Spinocerebellar ataxia Type 7), and TBP (Spinocerebellar ataxia Type 17).

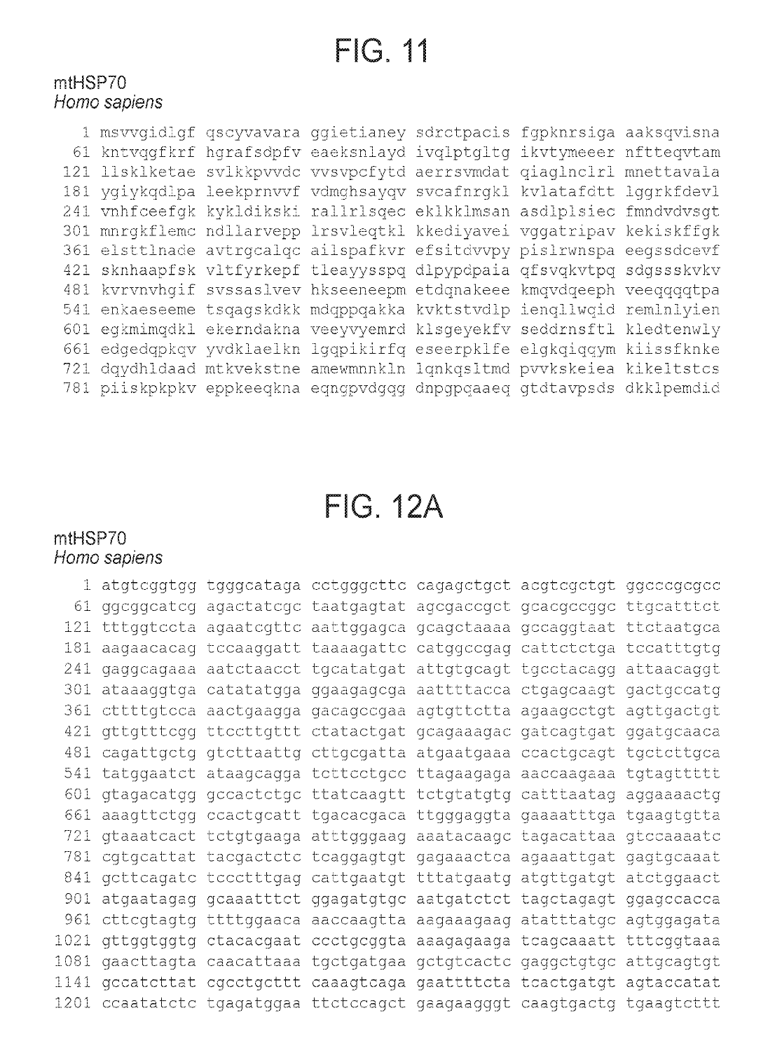

An mtHSP70 gene product is in some cases an mRNA that encodes an mtHSP70 polypeptide. An mtHSP70 gene product is in some cases an mtHSP70 polypeptide. An mtHSP70 polypeptide can comprise an amino acid sequence having at least 75%, at least 80%, at least 85%, at least 90%, at least 95%, at least 98%, at least 99%, or 100%, amino acid sequence identity to the amino acid sequence depicted in FIG. 11. See also, Mahalka et al. (2014) Biochim. Biophys. Acta 1838:1344; and Fathallah et al. (1993) J. Immunol. 151:810.

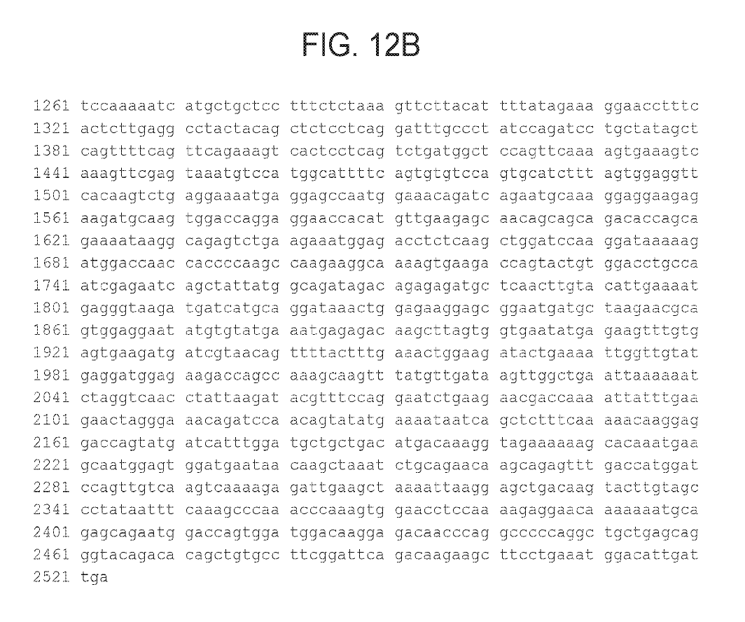

A nucleic acid agent that reduces the level of an mtHSP70 gene product can be readily designed using a known mtHSP70 nucleotide sequence. For example, a nucleic acid agent that reduces the level of an mtHSP70 gene product can be readily designed using the nucleotide sequence depicted in FIG. 12A-12B.

### CPT Inhibitors

Suitable CPT inhibitors include, but are not limited to, heteroaryl substituted piperidine derivatives (EP 1959951 B1), CPT-1 inhibitor ST1326 (WO 2009/002433), piperidine-amide derivatives (WO 2008/145596), sulfonamide derivatives (WO 2008/074692), oxirane carboxylate and other compounds (WO 2006/041922), trimetazidine and perhexiline derivatives (WO 2007/096251), sulfonamide derivatives (WO 2006/131452), bicyclic sulfonamide derivatives (U.S. 2007/0191603), indolyl derivatives (U.S. 2007/0060567), 4-trimethylammonio-butyrates (U.S. 2009/0270500), aminobutanoic acid derivatives (WO 2006/092204), malonyl-CoA, adriamycin; D,L-aminocarnitine; acylamino carnitines; decanoylcarnitine; amiodarone; 2-bromopalmitic acid; 2-bromopalmitoylcarnitine; 2-bromopalmitoyl-CoA; 2-bromomyristoylthiocarnitine; emeriamine; erucic acid; erucylcarnitine; etomoxir (Ethyl 2-[6-(4-chlorophenoxy)hexyl]oxirane-2-carboxylate); etomoxiryl-CoA; glyburide; hemiacetylcarnitinium chloride; hemipalmitoylcanitinium chloride; 3-hydroxy-5-5-dimethylhexanoic acid (HDH); methyl palmoxirate(methyl-2-tetradecylglycidate); 2-tetradecylglycidic acid; oxfenicine ((2S)-2-amino-2-(4-hydroxyphenyl) ethanoic acid); perhexiline (2-(2,2-dicyclohexylethyl)piperidine); 2[5(4-chloropheyl)pentyl]-oxirane-2-carboxylic acid (POCA); 2-[3-(3-trifluoromethylphenyl)-propyl]oxiran-2-carbonyl-CoA; 2-[5-(4-chlorophenyl)pentyl]-oxiran-2-carbonyl-CoA; 2-(5-phenylpentyl)oxiran-2-carbonyl-CoA; 2-tetradecyloxiran-2-carbonyl-CoA; mildronate (2-(2-Carboxylato-ethyl)-1,1,1-trimethylhydrazinium); (8,N,N-diethylamino-octyl-3,4,5-trimethoxybenzoate (TMB-8); tolbutamide; and trimetazidine. The CPT inhibitors disclosed in these references are hereby incorporated by reference. In some cases, the CPT inhibitors exclude any of the foregoing.

### Nucleic Acid Agents

In some cases, an agent is a nucleic acid agent. In some cases, a nucleic acid agent reduces the level of mitochondrial heat shock protein 70 (mtHSP70) in a cell.

Suitable agents that reduce the level of mtHSP70 in a cell include interfering nucleic acids, e.g., interfering RNA molecules. In one embodiment, reduction of the level of mtHSP70 is accomplished through RNA interference (RNAi) by contacting a cell with a small nucleic acid molecule, such as a short interfering nucleic acid (siNA), an antisense RNA, a short interfering RNA (siRNA), a double-stranded RNA (dsRNA), a micro-RNA (miRNA), or a short hairpin RNA (shRNA) molecule, or modulation of expression of a small interfering RNA (siRNA) so as to provide for decreased levels of mtHSP70.

In some cases, an agent is an RNA or a DNA molecule. In some cases, a nucleic acid agent is an RNAi agent (for example, miRNAs, siRNAs, shRNAs, antisense oligonucleotides, ribozymes). In some cases, a nucleic acid agent is a gene therapy vector.

RNA interference or RNAi refers to sequence-specific inhibition of gene expression and/or reduction in target RNA levels mediated by an at least partly double-stranded RNA, which RNA comprises a portion that is substantially complementary to a target RNA. Typically, at least part of the substantially complementary portion is within the double stranded region of the RNA. In some embodiments, RNAi can occur via selective intracellular degradation of RNA. In some embodiments, RNAi can occur by translational repression.

An RNAi agent is an RNA, optionally including one or more nucleotide analogs or modifications, having a structure characteristic of molecules that can mediate inhibition of gene expression through an RNAi mechanism. In some embodiments, RNAi agents mediate inhibition of gene expression by causing degradation of target transcripts. In some embodiments, RNAi agents mediate inhibition of gene expression by inhibiting translation of target transcripts. Generally, an RNAi agent includes a portion that is substantially complementary to a target RNA. In some embodiments, RNAi agents are at least partly double-stranded. In some embodiments, RNAi agents are single-stranded. In some embodiments, exemplary RNAi agents can include siRNA, shRNA, and/or miRNA. In some embodiments, RNAi agents may be composed entirely of natural RNA nucleotides (i.e., adenine, guanine, cytosine, and uracil). In some embodiments, RNAi agents may include one or more non-natural RNA nucleotides (e.g., nucleotide analogs, DNA nucleotides, etc.). Inclusion of non-natural RNA nucleic acid residues may be used to make the RNAi agent more resistant to cellular degradation than RNA. In some embodiments, the term “RNAi agent” may refer to any RNA, RNA derivative, and/or nucleic acid encoding an RNA that induces an RNAi effect (e.g., degradation of target RNA and/or inhibition of translation). In some embodiments, an RNAi agent may comprise a blunt-ended (i.e., without overhangs) dsRNA that can act as a Dicer substrate. For example, such an RNAi agent may comprise a blunt-ended dsRNA which is >25 base pairs length, which may optionally be chemically modified to abrogate an immune response.

The terms microRNA or miRNA refer to an RNAi agent that is approximately 21-23 nucleotides (nt) in length. miRNAs can range between 18-26 nucleotides in length. Typically, miRNAs are single-stranded. However, in some embodiments, miRNAs may be at least partially double-stranded. In certain embodiments, miRNAs may comprise an RNA duplex (referred to herein as a “duplex region”) and may optionally further comprises one or two single-stranded overhangs. In some embodiments, an RNAi agent comprises a duplex region ranging from 15 to 29 by in length and optionally further comprising one or two single-stranded overhangs. An miRNA may be formed from two RNA molecules that hybridize together, or may alternatively be generated from a single RNA molecule that includes a self-hybridizing portion. In general, free 5′ ends of miRNA molecules have phosphate groups, and free 3′ ends have hydroxyl groups. The duplex portion of an miRNA usually, but does not necessarily, comprise one or more bulges consisting of one or more unpaired nucleotides. One strand of an miRNA includes a portion that hybridizes with a target RNA. In some cases, one strand of the miRNA is not precisely complementary with a region of the target RNA, meaning that the miRNA hybridizes to the target RNA with one or more mismatches. In some case, one strand of the miRNA is precisely complementary with a region of the target RNA, meaning that the miRNA hybridizes to the target RNA with no mismatches. Typically, miRNAs are thought to mediate inhibition of gene expression by inhibiting translation of target transcripts. However, in some embodiments, miRNAs may mediate inhibition of gene expression by causing degradation of target transcripts.

The term “short, interfering RNA” (or “siRNA”) refers to an RNAi agent comprising an RNA duplex (referred to herein as a “duplex region”) that is approximately 19 basepairs (bp) in length and optionally further comprises one or two single-stranded overhangs. In some embodiments, an RNAi agents comprises a duplex region ranging from 15 to 29 by in length and optionally further comprising one or two single-stranded overhangs. An siRNA may be formed from two RNA molecules that hybridize together, or may alternatively be generated from a single RNA molecule that includes a self-hybridizing portion. In general, free 5′ ends of siRNA molecules have phosphate groups, and free 3′ ends have hydroxyl groups. The duplex portion of an siRNA may, but typically does not, comprise one or more bulges consisting of one or more unpaired nucleotides. One strand of an siRNA includes a portion that hybridizes with a target RNA. In some cases, one strand of the siRNA is precisely complementary with a region of the target RNA, meaning that the siRNA hybridizes to the target RNA without a single mismatch. In some cases, one or more mismatches between the siRNA and the targeted portion of the target RNA may exist. In some cases, in which perfect complementarity is not achieved, any mismatches are generally located at or near the siRNA termini. In some cases, siRNAs mediate inhibition of gene expression by causing degradation of target transcripts.

The term “short hairpin RNA” (or “shRNA”) refers to an RNAi agent comprising an RNA having at least two complementary portions hybridized or capable of hybridizing to form a double-stranded (duplex) structure sufficiently long to mediate RNAi (typically at least approximately 19 by in length), and at least one single-stranded portion, typically ranging between approximately 1 and 10 nucleotides (nt) in length that forms a loop. In some embodiments, an shRNA comprises a duplex portion ranging from 15 to 29 by in length and at least one single-stranded portion, typically ranging between approximately 1 and 10 nt in length that forms a loop. The duplex portion may, but typically does not, comprise one or more bulges consisting of one or more unpaired nucleotides. In some embodiments, siRNAs mediate inhibition of gene expression by causing degradation of target transcripts. shRNAs are thought to be processed into siRNAs by the conserved cellular RNAi machinery. Thus shRNAs may be precursors of siRNAs. Regardless, siRNAs in general are capable of inhibiting expression of a target RNA, similar to siRNAs.

Certain nucleic acid molecules, referred to as ribozymes or deoxyribozymes, have been shown to catalyze the sequence-specific cleavage of RNA molecules. The cleavage site is determined by complementary pairing of nucleotides in the RNA or DNA enzyme with nucleotides in the target RNA. Thus, RNA and DNA enzymes can be designed to cleave to any RNA molecule, thereby increasing its rate of degradation (Cotten et al, EMBO J. 8: 3861, 1989; Usman et al., Nucl. Acids Mol. Biol. 10: 243, 1996; Usman, et al., Curr. Opin. Struct. Biol. 1: 527, 1996; Sun, et al., Pharmacol. Rev., 52: 325, 2000. See also e.g., Cotten et al, EMBO J. 8: 3861, 1989).

The present disclosure further provides a nucleic acid (including an expression vector) that comprises a nucleotide sequence that encodes a subject nucleic acid agent (e.g., an antisense; an siNA; etc.). Suitable expression vectors include, e.g., a viral vector. In some embodiments, the nucleic acid agent-encoding nucleotide sequence is operably linked to a keratinocyte-specific promoter. In some embodiments, the nucleic acid agent-encoding nucleotide sequence is operably linked to an inducible promoter. In the discussion herein relating to compositions comprising, and methods involving use of, a nucleic acid agent, it should be understood that the present disclosure contemplates compositions comprising a nucleic acid comprising a nucleotide sequence that encodes a subject nucleic acid agent, and methods involving use of a nucleic acid comprising a nucleotide sequence that encodes a subject nucleic acid agent.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

### Modifications

In some embodiments, a subject nucleic acid (e.g., an siRNA; an antisense nucleic acid) comprises one or more modifications, e.g., a base modification, a backbone modification, etc. As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound; however, linear compounds are generally suitable. In addition, linear compounds may have internal nucleotide base complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

### Modified Backbones and Modified Internucleoside Linkages

Examples of suitable modifications include modified backbones or non-natural internucleoside linkages. Nucleic acids (e.g., a subject siRNA; a subject antisense nucleic acid) having modified backbones include those that retain a phosphorus atom in the backbone and those that do not have a phosphorus atom in the backbone.

Suitable modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Suitable oligonucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts (such as, for example, potassium or sodium), mixed salts and free acid forms are also included.

In some embodiments, a subject nucleic acid (e.g., a subject siRNA; a subject antisense nucleic acid) comprises one or more phosphorothioate and/or heteroatom internucleoside linkages, in particular —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—). MMI type internucleoside linkages are disclosed in the above referenced U.S. Pat. No. 5,489,677. Suitable amide internucleoside linkages are disclosed in t U.S. Pat. No. 5,602,240.

Also suitable are nucleic acids (e.g., a subject siRNA; a subject antisense nucleic acid) having morpholino backbone structures as described in, e.g., U.S. Pat. No. 5,034,506. For example, in some embodiments, a subject nucleic acid (e.g., a subject antisense nucleic acid; a subject synthetic target protector nucleic acid) comprises a 6-membered morpholino ring in place of a ribose ring. In some of these embodiments, a phosphorodiamidate or other non-phosphodiester internucleoside linkage replaces a phosphodiester linkage.

Suitable modified polynucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

### Mimetics

A subject nucleic acid (e.g., a subject siRNA; a subject antisense nucleic acid) can be a nucleic acid mimetic. The term “mimetic” as it is applied to polynucleotides is intended to include polynucleotides wherein only the furanose ring or both the furanose ring and the internucleotide linkage are replaced with non-furanose groups, replacement of only the furanose ring is also referred to in the art as being a sugar surrogate. The heterocyclic base moiety or a modified heterocyclic base moiety is maintained for hybridization with an appropriate target nucleic acid. One such nucleic acid, a polynucleotide mimetic that has been shown to have excellent hybridization properties, is referred to as a peptide nucleic acid (PNA). In PNA, the sugar-backbone of a polynucleotide is replaced with an amide containing backbone, in particular an aminoethylglycine backbone. The nucleotides are retained and are bound directly or indirectly to aza nitrogen atoms of the amide portion of the backbone.

One polynucleotide mimetic that has been reported to have excellent hybridization properties is a peptide nucleic acid (PNA). The backbone in PNA compounds is two or more linked aminoethylglycine units which gives PNA an amide containing backbone. The heterocyclic base moieties are bound directly or indirectly to aza nitrogen atoms of the amide portion of the backbone. Representative U.S. patents that describe the preparation of PNA compounds include, but are not limited to: U.S. Pat. Nos. 5,539,082; 5,714,331; and 5,719,262.

Another class of polynucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. One class of linking groups has been selected to give a non-ionic oligomeric compound. The non-ionic morpholino-based oligomeric compounds are less likely to have undesired interactions with cellular proteins. Morpholino-based polynucleotides are non-ionic mimics of oligonucleotides which are less likely to form undesired interactions with cellular proteins (Dwaine A. Braasch and David R. Corey, Biochemistry, 2002, 41(14), 4503-4510). Morpholino-based polynucleotides are disclosed in U.S. Pat. No. 5,034,506. A variety of compounds within the morpholino class of polynucleotides have been prepared, having a variety of different linking groups joining the monomeric subunits.

A further class of polynucleotide mimetic is referred to as cyclohexenyl nucleic acids (CeNA). The furanose ring normally present in an DNA/RNA molecule is replaced with a cyclohenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (see Wang et al., J. Am. Chem. Soc., 2000, 122, 8595-8602). In general the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation.

A further modification includes Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage can be a methylene (—CH₂—), group bridging the 2′ oxygen atom and the 4′ carbon atom wherein n is 1 or 2 (Singh et al., Chem. Commun., 1998, 4, 455-456). LNA and LNA analogs display very high duplex thermal stabilities with complementary DNA and RNA (Tm=+3 to +10 C), stability towards 3′-exonucleolytic degradation and good solubility properties. Potent and nontoxic antisense oligonucleotides containing LNAs have been described (Wahlestedt et al., Proc. Natl. Acad. Sci. U.S.A., 2000, 97, 5633-5638).

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

### Modified Sugar Moieties

A subject nucleic acid (e.g., a subject siNA; a subject antisense nucleic acid) can also include one or more substituted sugar moieties. Suitable polynucleotides comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C.sub.1 to C₁₀ alkyl or C₂ to C₁₀ alkenyl and alkynyl. Particularly suitable are O((CH₂)ₙO)ₘCH₃, O(CH₂)ₙOCH₃, O(CH₂)ₙNH₂, O(CH₂)ₙCH₃, O(CH₂)—ONH₂, and O(CH₂)—ON((CH₂)—CH₃)₂, where n and m are from 1 to about 10. Other suitable polynucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A suitable modification includes 2′-methoxyethoxy (2′-O—CH₂ CH₂OCH₃, also known as 2′-O-(2-methoxyethyl) or 2′-MOE) (Martin et al., Helv. Chim. Acta, 1995, 78, 486-504) i.e., an alkoxyalkoxy group. A further suitable modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Other suitable sugar substituent groups include methoxy (—O—CH₃), aminopropoxy (—O CH₂ CH₂ CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar.

### Base Modifications and Substitutions

A subject nucleic acid (e.g., a subject siRNA; a subject antisense nucleic acid) may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′:4,5)pyrrolo(2,3-d)pyrimidin-2-one).

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are useful for increasing the binding affinity of an oligomeric compound (e.g., an antisense nucleic acid; a target protector nucleic acid). These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi et al., eds., Antisense Research and Applications, CRC Press, Boca Raton, 1993, pp. 276-278) and are suitable base substitutions, e.g., when combined with 2′-O-methoxyethyl sugar modifications.

### Conjugates

Another possible modification of a subject nucleic acid (e.g., a subject siRNA; a subject antisense nucleic acid) involves chemically linking to the polynucleotide one or more moieties or conjugates which enhance the activity, cellular distribution or cellular uptake of the oligonucleotide. These moieties or conjugates can include conjugate groups covalently bound to functional groups such as primary or secondary hydroxyl groups. Conjugate groups include, but are not limited to, intercalators, reporter molecules, polyamines, polyamides, polyethylene glycols, polyethers, groups that enhance the pharmacodynamic properties of oligomers, and groups that enhance the pharmacokinetic properties of oligomers. Suitable conjugate groups include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that enhance the pharmacokinetic properties include groups that improve uptake, distribution, metabolism or excretion of a subject antisense nucleic acid or target protector nucleic acid.

Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937.

### Treatment Methods

The present disclosure provides methods of treating a disease or disorder associated with protein misfolding and/or aggregation. The methods generally involve administering to an individual in need thereof an effective amount of a CPT inhibitor and/or an effective amount of a nucleic acid agent that reduces the level of an mtHSP70 gene product.

In some cases, an “effective amount” of an active agent (e.g., a CPT inhibitor and/or an effective amount of a nucleic acid agent that reduces the level of an mtHSP70 gene product) is an amount that reduces the level of protein aggregates in a cell, extracellular fluid, tissue, or organ in an individual, by at least 10%, at least 20%, at least 30%, at least 40%, at least 50%, at least 60%, at least 70%, at least 80%, or more than 80%, compared to the level of protein aggregates in the cell extracellular fluid, tissue, or organ in the individual in the absence of treatment with the active agent.

Proteotoxicity-associated diseases and disorders that can be treated using a method of the present disclosure include diseases and disorders characterized by increased aggregation-associated proteotoxicity. In such diseases, aggregation exceeds clearance inside and/or outside of the cell. Proteotoxicity-associated diseases can be associated with aging. Exemplary proteotoxicity-associated diseases include, but are not limited to, neurodegenerative diseases associated with aggregation of polyglutamine repeats in proteins or repeats at other amino acids such as alanine; Lewy body diseases, and other disorders associated with α-synuclein aggregation; amyotrophic lateral sclerosis; transthyretin-associated aggregation diseases; Alzheimer's disease; age-associated macular degeneration; inclusion body myositosis; and prion diseases. Neurodegenerative diseases associated with aggregation of polyglutamine include, but are not limited to, Huntington's disease, dentatorubral and pallidoluysian atrophy, several forms of spinocerebellar ataxia, and spinal and bulbar muscular atrophy. Alzheimer's disease is characterized by the formation of two types of aggregates: intracellular and extracellular aggregates of AP peptide and intracellular aggregates of the microtubule associated protein tau. Transthyretin-associated aggregation diseases include, for example, senile systemic amyloidoses, familial amyloidotic neuropathy, and familial amyloid cardiomyopathy. Lewy body diseases are characterized by an aggregation of α-synuclein protein and include, for example, Parkinson's disease. Prion diseases (also known as transmissible spongiform encephalopathies) are characterized by aggregation of prion proteins. Exemplary human prion diseases are Creutzfeldt-Jakob Disease (CJD), Variant Creutzfeldt-Jakob Disease, Gerstmann-Straussler-Scheinker Syndrome, Fatal Familial Insomnia, and Kuru.

In some cases, a proteotoxicity-associated disorder is a proteinopathy. As used herein, the term “proteinopathy” or “proteinopathic” refers to a disease, disorder, and/or condition associated with the pathogenic aggregation and/or accumulation of one or more types of proteins, for example, but not limited to .alpha.-synuclein, Pamyloid, and/or tau proteins. In some cases, a proteinopathy is characterized by an anomaly in one or more of protein production, folding, aggregation, metabolism, or degradation (e.g. autophagy), transportation, etc. In some embodiments, proteinopathies are neurodegenerative diseases. In some embodiments, proteinopathies are inflammatory diseases. In some embodiments, proteinopathies are cardiovascular diseases. In some embodiments, proteinopathies are proliferative diseases. Specific pathologies such as synucleinopathies, tauopathies, amyloidopathies, TDP-43 proteinopathies and others are examples of proteinopathies. Exemplary proteins implicated in proteinopathies include: α-synuclein in the case of Parkinson's disease, Lewy body disease, and other synucleinopathies; tau and pamyloid in the case of Alzheimer's disease and certain other neurodegenerative diseases; SOD1 and TDP-43 in the case of amyotrophic lateral sclerosis; huntingtin in the case of Huntington's disease; rhodopsin in the case of retinitis pigmentosa; and proteins involved in lysosomal storage diseases.

### Compositions and Formulations

In carrying out a method of the present disclosure for treating a disease or disorder associated with protein misfolding and/or aggregation, a compositions, e.g., pharmaceutical compositions, comprising an agent, as described above, is administered to an individual in need thereof. The present disclosure provides compositions, e.g., pharmaceutical compositions, comprising an agent that inhibits CPT activity. The present disclosure provides compositions, e.g., pharmaceutical compositions, comprising an agent that reduces the level of mtHSP70 in a cell. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

A composition can include: a) an active agent (e.g., a CPT inhibitor; a nucleic acid that reduces the level of mtHSP70); and b) one or more of: a buffer, a surfactant, an antioxidant, a hydrophilic polymer, a dextrin, a chelating agent, a suspending agent, a solubilizer, a thickening agent, a stabilizer, a bacteriostatic agent, a wetting agent, and a preservative. Suitable buffers include, but are not limited to, (such as N,N-bis(2-hydroxyethyl)-2-aminoethanesulfonic acid (BES), bis(2-hydroxyethyl)amino-tris(hydroxymethyl)methane (BIS-Tris), N-(2-hydroxyethyl)piperazine-N′3-propanesulfonic acid (EPPS or HEPPS), glycylglycine, N-2-hydroxyehtylpiperazine-N′-2-ethanesulfonic acid (HEPES), 3-(N-morpholino)propane sulfonic acid (MOPS), piperazine-N,N′-bis(2-ethane-sulfonic acid) (PIPES), sodium bicarbonate, 3-(N-tris(hydroxymethyl)-methyl-amino)-2-hydroxy-propanesulfonic acid) TAPSO, (N-tris(hydroxymethyl)methyl-2-aminoethanesulfonic acid (TES), N-tris(hydroxymethyl)methyl-glycine (Tricine), tris(hydroxymethyl)-aminomethane (Tris), etc.). Suitable salts include, e.g., NaCl, MgCl₂, KCl, MgSO₄, etc.

A pharmaceutical formulation can include: a) an active agent (e.g., a CPT inhibitor; a nucleic acid that reduces the level of mtHSP70); and b) a pharmaceutically acceptable excipient.

A pharmaceutical formulation, which may conveniently be presented in unit dosage form, can be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

An active agent (e.g., a CPT inhibitor; a nucleic acid that reduces the level of mtHSP70) can be formulated into any of many possible dosage forms such as, but not limited to, tablets, capsules, gel capsules, liquid syrups, soft gels, suppositories, and enemas. An active agent (e.g., a CPT inhibitor; a nucleic acid that reduces the level of mtHSP70) can also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

A pharmaceutical composition comprising an active agent (e.g., a CPT inhibitor; a nucleic acid that reduces the level of mtHSP70) may include solutions, emulsions, foams and liposome-containing formulations. A pharmaceutical composition comprising an active agent (e.g., a CPT inhibitor; a nucleic acid that reduces the level of mtHSP70) can comprise one or more penetration enhancers, carriers, excipients, or other active or inactive ingredients.

Emulsions are typically heterogeneous systems of one liquid dispersed in another in the form of droplets, which can exceed 0.1 μm in diameter. Emulsions may contain additional components in addition to the dispersed phases, and the active agent (e.g., a CPT inhibitor; a nucleic acid agent) which can be present as a solution in the aqueous phase, the oily phase, or as a separate phase. Microemulsions are also suitable. Emulsions and their uses are well known in the art and are further described in U.S. Pat. No. 6,287,860.

A pharmaceutical formulation comprising an active agent (e.g., a CPT inhibitor; a nucleic acid that reduces the level of mtHSP70) can be a liposomal formulation. As used herein, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes that can interact with negatively charged DNA molecules to form a stable complex. Liposomes that are pH sensitive or negatively charged are believed to entrap DNA rather than complex with it. Both cationic and noncationic liposomes can be used to deliver a nucleic acid agent.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860, which is incorporated herein by reference in its entirety.

The formulations and compositions of the present disclosure may also include surfactants. The use of surfactants in drug products, formulations and in emulsions is well known in the art. Surfactants and their uses are further described in U.S. Pat. No. 6,287,860.

In one embodiment, various penetration enhancers are included, to effect the efficient delivery of an agent. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860, which is incorporated herein by reference in its entirety.

A nucleic acid agent can be conjugated to poly(L-lysine) to increase cell penetration. Such conjugates are described by Lemaitre et al., Proc. Natl. Acad. Sci. USA, 84, 648-652 (1987). The procedure requires that the 3′-terminal nucleotide be a ribonucleotide. The resulting aldehyde groups are then randomly coupled to the epsilon-amino groups of lysine residues of poly(L-lysine) by Schiff base formation, and then reduced with sodium cyanoborohydride.

One of skill in the art will recognize that formulations are routinely designed according to their intended use and/or route of administration.

Suitable formulations for topical administration include those in which a subject agent is in admixture with a topical delivery agent such as lipids, liposomes, fatty acids, fatty acid esters, steroids, chelating agents and surfactants. Suitable lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA).

For topical or other administration, an active agent can be encapsulated within liposomes or may form complexes thereto, in particular to cationic liposomes. Alternatively, an active agent (e.g., a nucleic acid agent) can be complexed to lipids, in particular to cationic lipids. Suitable fatty acids and esters, pharmaceutically acceptable salts thereof, and their uses are further described in U.S. Pat. No. 6,287,860.

Compositions and formulations for oral administration include powders or granules, microparticulates, nanoparticulates, suspensions or solutions in water or non-aqueous media, capsules, gel capsules, sachets, tablets, or minitablets. Thickeners, flavoring agents, diluents, emulsifiers, dispersing aids or binders may be desirable. Suitable oral formulations include those in which an agent is administered in conjunction with one or more penetration enhancers surfactants and chelators. Suitable surfactants include, but are not limited to, fatty acids and/or esters or salts thereof, bile acids and/or salts thereof. Suitable bile acids/salts and fatty acids and their uses are further described in U.S. Pat. No. 6,287,860. Also suitable are combinations of penetration enhancers, for example, fatty acids/salts in combination with bile acids/salts. An exemplary suitable combination is the sodium salt of lauric acid, capric acid, and UDCA. Further penetration enhancers include, but are not limited to, polyoxyethylene-9-lauryl ether, and polyoxyethylene-20-cetyl ether. Suitable penetration enhancers also include propylene glycol, dimethylsulfoxide, triethanoiamine, N,N-dimethylacetamide, N,N-dimethylformamide, 2-pyrrolidone and derivatives thereof, tetrahydrofurfuryl alcohol, and AZONE™.

An active agent can be delivered orally, in granular form including sprayed dried particles, or complexed to form micro or nanoparticles.

Compositions and formulations for enteral or parenteral administration may include sterile aqueous solutions which may also contain buffers, diluents and other suitable additives such as, but not limited to, penetration enhancers, carrier compounds and other pharmaceutically acceptable carriers or excipients. In some cases, the formulation is one that is suitable for topical application to the skin.

### Delivery and Routes of Administration

An agent can be administered by any suitable means. One skilled in the art will appreciate that many suitable methods of administering an agent to a host in the context of the present disclosure, e.g., a human, are available, and, although more than one route may be used to administer a particular nucleic acid agent, a particular route of administration may provide a more immediate and more effective reaction than another route.

Suitable routes of administration include enteral and parenteral routes. Administration can be via a local or a systemic route of administration. An agent can be administered in a number of ways depending upon whether local or systemic treatment is desired and upon the area to be treated. Administration may be topical, pulmonary, e.g., by inhalation or insufflation of powders or aerosols, including by nebulizer; intratracheal, intranasal, epidermal and transdermal), oral or parenteral. Parenteral administration includes, but is not limited to, intravenous, intraarterial, subcutaneous, intraperitoneal, or intramuscular injection or infusion.

In some embodiments, an agent is administered topically to the skin. In other embodiments, an agent is administered intradermally. In other embodiments, an agent is administered subcutaneously. In some embodiments, an agent is administered intramuscularly. In some embodiments, an agent is administered orally. In some embodiments, an agent is administered intravenously.

### Dosing

The formulation of therapeutic compositions and their subsequent administration (dosing) is within the skill of those in the art. Dosing is dependent on several criteria, including severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual agents, and can generally be estimated based on EC50s found to be effective in vitro and in vivo animal models.

For example, a suitable dose of an agent can be from 0.01 μg to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, or from 100 μg to 1 mg per kg of body weight. Persons of ordinary skill in the art can easily estimate repetition rates for dosing based on measured residence times and concentrations of the agent in bodily fluids or tissues. Following successful treatment, it may be desirable to have the patient undergo maintenance therapy, wherein an agent is administered in maintenance doses, ranging from 0.01 μg to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, or from 100 μg to 1 mg per kg of body weight.

In some embodiments, multiple doses of an agent are administered. The frequency of administration of an agent can vary depending on any of a variety of factors. For example, in some embodiments, an agent is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

The duration of administration of an agent, e.g., the period of time over which an active agent is administered, can vary, depending on any of a variety of factors, e.g., patient response, etc. For example, an active agent can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Subjects suitable for treatment with a method of the present disclosure include individuals who have been diagnosed as having a disease or disorder associated with protein misfolding and/or aggregation. Subjects suitable for treatment with a method of the present disclosure include individuals who have been treated for a disease or disorder associated with protein misfolding and/or aggregation; and who have failed to respond to such treatment.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

The following materials and methods were used in the Examples described below.

### Strains

SJ4100 (zcIs13[hsp-6p::GFP]), SJ4058 (zcIs9[hsp-60p::GFP]), CL2070 (dvIs[hsp-16.2p::GFP]), SJ4500 (zcIs4[hsp-4p::GFP]), (CF512 (fer-15(b26);fem-1(hc17)), AM140 (rm1s132[unc-54p::Q35::YFP]), SJ4197 (zcIs39 [dve-1p::dve-1::GFP]), AGD710 (N2, uth1s235 [sur-5p::hsf-1, myo-2p::tomato] and N2 wild type were obtained from the Caenorhabditis Genetics Center. CL2070 strain was crossed with CF512 strain to generate a temperature-sensitive sterile reporter strain AGD919 (dvIs[hsp-16.2p::GFP]; fer-15(b26);fem-1(hc17)). RNAi screenings were done with AGD919.

### RNAi Treatment and Quantification of GFP Induction

Bacterial feeding of RNAi experiments was conducted during adulthood to exclude target genes' function during developmental stages. Synchronized eggs were harvested by bleaching and nematodes were grown on plates with E. coli OP50 until they reached early adulthood before they were transferred to RNAi plates. Day 1 adult worms were then grown on the RNAi plates with E. coli HT115 that carried the RNAi construct for 3 days at 20° C. Worms were imaged using a fluorescent microscope for green fluorescent protein (GFP) induction or applied to COPAS Biosorter (Union Biometrica) to quantify the level of GFP induction. The temperature-sensitive sterile strains were grown at 15° C. until they were gravid adults. Then, the cohorts were shifted to 25° C. Day 1 adult worms were transferred to RNAi plates for 2 days before images were taken. For the double RNAi treatment, E. coli carrying the indicated RNAi constructs are mixed 1:1 ratio.

### RNAi Screening (Mitochondrial Import Machinery Components)

A list of genes for RNAi were obtained from a previous study (Ichishita et al., J. Biochem. 2008, 143:449-454). AGD919 (dvIs[hsp-16. 2p::GFP]; fer-15(b26);fem-1(hc17)) eggs were synchronized by bleaching and grown on E. coli OP50 plates until they reached day 1 adult at 25° C. Day 1 adult worms were transferred to E. coli HT115 RNAi plates and grown for 2 more days before the analysis. Then, GFP induction was measured using ImageExpress (Molecular devices) to identify worms with more than a 2-fold increase in GFP expression compared to the control worms. All experiments were independently repeated three times.

### Microarray Analysis

Synchronized N2 worms were grown on E. coli HT115 RNAi plates from day 1 adult for 3 days. Plates were washed-off with M9 every other day to get rid of eggs and larvae. Worms were harvested after 3 days on RNAi plates to isolate RNA for microarray. Raw expression data files were obtained for three replicates each of N2 worms treated with hsp-6 RNAi, hsp-6/hsf-1 RNAi, hsp-6/dve-1 RNAi and empty vector (EV) with the Affymetrix C. elegans Genome Array. All microarray analysis was performed with Bioconductor. Briefly, standard data quality validation as suggested by Affymetrix was carried out with the ‘simpleaffy’ package, followed by ‘affyPLM’, which identified no problematic chips. The raw data were preprocessed according to the GCRMA method (implemented in ‘gcrma’), which performs probe-sequence-based background adjustment, quantile normalization, and utilizes a robust multi-chip average to summarize information into single expression measurements for each probeset. Before statistical testing, the data were submitted to a non-specific filter (via the package ‘genefilter’) that removed probesets with an expression interquartile range smaller than 0.5. To identify genes that were significantly differentially expressed between conditions, linear modeling and empirical Bayes analysis was performed using the ‘limma’ package. Limma computes an empirical Bayes adjustment for the t-test (moderated t-statistic), which is more robust than the standard two-sample t-test comparisons. To correct for multiple testing, Benjamin and Hochberg's method to control for false discovery rate was used. Genes with an adjusted p-value of 0.05 or smaller and a fold-change in expression larger than two-fold were considered differentially expressed. Ward's minimum variance method was used to cluster normalized expression values for genes differentially expressed in hsp-6 RNAi. Genes from selected clusters were submitted to DAVID to identify statistically over-represented function annotations.

### Functional Enrichment Testing

Microarray analysis expression data was used to test for enriched Gene Ontology Biological Process terms (Ashburner et al., 2000) with LRPath (Sartor et al., 2009; Bioinforma. Oxf. Engl. 25, 211-217), a logisitic regression-based gene set enrichment method. LRpath relates the odds of gene set membership with the significance of differential expression (p-values from limma). GO terms with an FDR of less than 1e-03 were deemed significant. Directional LRpath tests were used to distinguish between upregulated and downregulated terms. First, GO terms enriched in hsp-6 RNAi versus EV comparison were identified. Then, GO terms dependent on DVE-1 and HSF-1 were identified as those enriched in hsp-6; dve-1 RNAi versus EV; hsp-6 RNAi and hsp-6; hsf-1 RNAi vs EV; hsp-6 RNAi comparisons but with opposite regulation pattern. Representative GO terms were identified by clustering similar terms semantically with REVIGO (Supek et al., 2011), using a similarity cutoff (SimRel) of 0.5.

### Quantitative Polymerase Chain Reaction (QPCR)

Total RNA was harvested from at least 500 worms using Qiazol reagent (QIAGEN). RNA was purified using an RNeasy mini column (QIAGEN), then cDNA was synthesized using the QuantiTect reverse transcription kit (QIAGEN). SybrGreen quantitative reverse transcription-polymerase chain reaction (RT-PCR) experiments were performed according to the manufacturer's manual using an ABI Prism7900HT (Applied Biosystems), and data were analyzed using the comparative 2ΔΔCₜ method. pmp-3 and cdc-42 were used as housekeeping control genes for the analysis. Experiments were done with three biological repeats.

siRNA Transfection

The following siRNAs were tested for depleting the indicated genes in the human primary fibroblasts or HEK cells: hspa9 siRNA (#1 and #2), accl siRNA (#1 and #2), fas siRNA (#1 and #2) (Ambion). Scrambled siRNA with no known mammalian homology (non-targeting siRNA #1 (Ambion) was used as negative control. Double siRNA treatment was performed by mixing two different siRNAs indicated at 1:1 ratio. Cells were transfected with the siRNAs using JetPrime according to the manufacturer's manual and then harvested after 48 h. Control vector-transfected cells were used as controls for all the experiments.

### Mitochondrial Fractionation

8,000-10,000 animals were treated with indicated RNAi and washed off from plates. Worms were homogenized in mitochondrial isolation buffer (210 mM Mannitol, 70 mM Sucrose, 0.1 mM EDTA, 5 mM Tris-HCl, pH 7.4, and 1× protease inhibitor cocktail). Worm debris and nuclei were eliminated by centrifuging lysates for 15 min at 800 g. Supernatants containing mitochondria were then pelleted for 15 min at 12,000 g (supernatants were saved for cytosolic fractions). The mitochondrial pellet was washed with mitochondrial isolation buffer three times. 40 μg of proteins from each fraction (mitochondria and cytosol) was loaded onto the SDS-PAGE gel for Western blotting analysis.

### Sodium Dodecyl Sulfate (SDS)-Insoluble Protein Isolation

Isolation of SDS-insoluble protein from RNAi treated worms were performed as previously described with modifications (Reis-Rodrigues et al., Aging Cell. 2012, 11:120-127). Briefly, up to 5000 animals that were treated with RNAi were collected and washed with M9 media. Worm pellet was resuspended in phosphate-buffered saline (PBS) containing protease inhibitor cocktail (Roche) and sonicated on ice. Then the lysates were centrifuged for 10 min at 3000 g to remove cell debris. The same amount of proteins from each sample was then centrifuged for 15 min at 16,000 g and washed three times (saving the supernatants as PBS buffer-soluble proteins). The pellet was resuspended in PBS containing 1% SDS to extract SDS-soluble proteins and was centrifuged for 15 min at 16,000 g (saving the supernatants as SDS-soluble proteins). The pellets were then resuspended in 6 M GnHCl (60 min at 30° C.) to extract detergent-insoluble proteins. Samples were diluted and loaded onto the SDS-polyacrylamide gel electrophoresis (SDS-PAGE) gel for silver staining.

### Nile Red Staining and Nonyl Acridine Orange Staining

200-300 worms were washed off from plates with M9 for fixing. Briefly, worms were fixed with freshly made 0.5% paraformaldehyde and frozen in liquid nitrogen immediately. Worms underwent two freeze thaw cycles prior to complete thawing on ice and removal of the fixation solution. M9 with Nile Red was added (1 μg/ml in final concentration) to the worms prior to staining for 15-30 minutes. Worms were washed once with M9 before images were taken immediately. For the quantification of staining, we used COPAS biosorter using an RFP filter. For staining the cardiolipin contents, Nonyl Acridine Orange (NAO, Invitrogen) was used. After fixation and washing, 10 μM of NAO solution is added for 15-30 minutes. Prior to taking pictures (GFP filter), worms were washed with M9 once to remove extra NAO in the solution.

### Triglyceride Quantification

To quantify triglyceride content, a Triglyceride quantification kit (Bio Vision) was used according to the manufacturer's protocol. Briefly, worms were harvested from plates and washed with M9 three times. Worms were homogenized with 5% NP-40 in water using a glass dounce homogenizer to extract mostly the intestinal tissues by checking the lysates under a dissecting microscope. Worms looked like empty shells once the intestinal tissues were extracted out. The samples were heated slowly at 90° C. for 5 minutes and cooled down to room temperature. Heating was repeated once more prior to centrifugation of the samples for 2 minutes to bring down debris. Supernatant was removed and mixed with enzymes provided in the kit according to the manufacturer's manual. Measurement was done using microplate reader M1000 (TECAN) at Ex/Em=535/590 nm. Calculations were done after subtracting 0 standard readings as a background.

Sample Triglyceride control (C)=B/V×D nmol/μl or mM

Where: B is the amount of triglyceride from standard curve (nmol)

V is the sample volume added into the reaction well (μl)

D is the sample dilution factor

For the mammalian cell culture samples, cells were washed with cold PBS and homogenized with 5% NP-40 in water using a syringe with 22 G needle, followed by the procedure shown above. All experiments were done with three biological repeats.

### Motility Assay

Synchronized AM140 worms were transferred to the RNAi plates when they reached day 1 of adulthood. Worms on RNAi plates were washed-off with M9 to get rid of the eggs and larvae; worms were transferred to new plates every other day until they were day 4 adults. 15-20 worms were transferred to the M9 solution on an empty plate to take video for 30 seconds. Body bends were counted for 30 seconds for each worm; a total 12-15 worms were counted for body bends. All experiments were done in three biological repeats.

### Feeding Lipids to Worms

Indicated cardiolipins and ceramides were purchased from the Avanti Polar

Lipids, Inc. Lyophilized lipids were dissolved in Methanol with sonication. Ceramides were diluted to 100μg/ml and spotted on top of the OP50 plates or RNAi plates. Worms were transferred to Ceramide-spotted plates at L4 stage and the images were taken after 48 hours. Cardiolipin was fed to bacteria for 2-4 hours (OP50 or RNAi construct-carrying HT115) with final concentration of 100μg/ml. Then the bacteria were spotted on plates before transferring the worms. L4 worms were then transferred to the cardiolipin spotted plates and images were taken after 48 hours.

### Heat Shock Treatment

Worms were grown up to day 1 adults and underwent heat shock at 34° C. for 2 hours, then recovered at 20° C. for overnight before taking images. For the Nile Red Staining experiment, worms were recovered for 48 hours.

### Filter Trap Assay and Western Blotting Experiments

100μg of protein samples were applied on to cellulose acetate membrane with 0.22 μm pore size (Schlechtes+Schule), assembled in vacuum slot blotter (Bio-Dot, Bio-Rad). Membrane was washed with 0.2% SDS five times on the blotter and subjected to antibody incubation for detecting aggregated protein retained on the membrane. Briefly, membrane was incubated with anti-GFP antibody (1:3000 dilution in 5% milk in PBS, Roche) overnight in a cold room. Membrane was washed with PBST (PBST with 0.05% Tween-20) for three times, then incubated with secondary antibody (donkey anti-mouse antibody conjugated with HRP, 1:5000 dilution in 5% milk in PBS, Jackson Immuno Research). Membranes were washed with PBST three times and exposed to film after applying ECL solutions (Pierce) to visualize the protein bands.

For SDS-PAGE, 20-40μg of protein samples were loaded on 4-12% bis-tris SDS gel (Invitrogen). The gel was transferred to nitrocellulose membrane (GE) using XCell II blot module (Invitrogen). Then, membranes were incubated with antibodies and exposed to film as described above. mtHSP70 (Abcam), α-Tubulin (Sigma), GFP (Roche), and cytosolic HSP70 (HSP70/HSP72, Enzo life) were used to probe the membrane. The western blot bands' intensity was measured using ImageJ software.

### SDS-Insoluble Protein Isolation

Isolation of SDS-insoluble protein from RNAi treated worms were performed as previously described with modifications (Reis-Rodrigues et al., 2012; Aging Cell 11, 120-127). Briefly, up to 5000 animals that were treated with RNAi were collected and washed with M9 media. Worm pellet was resuspended in PBS containing protease inhibitor cocktail (Roche) and sonicated on ice. Then the lysates were centrifuged for 10 min at 3000 g to remove cell debris. The same amount of proteins from each sample was then centrifuged for 15 min at 16,000 g and washed three times (saving the supernatants as PBS buffer-soluble proteins). The pellet was resuspended in PBS containing 1% SDS to extract SDS-soluble proteins and was centrifuged for 15 min at 16,000 g (saving the supernatants as SDS-soluble proteins). The pellets were then resuspended in 6M GnHCl (60 min at 30° C.) to extract detergent-insoluble proteins. Samples were diluted and loaded onto the SDS-PAGE gel for silver staining.

### Proteasome Activity Assay

The in vitro assay of 26S proteasome activities was performed using a fluorogenic peptide substrate. Lysates were centrifuged at 10,000 g for 10 min at 4° C. Approximately 15-25μg of total protein of worm lysates were transferred to a 96-well microtiter plate (BD Falcon), and the fluorogenic substrate was then added to lysates. To measure the chymotrypsin-like activity of the proteasome we used Suc-Leu-Leu-Val-Tyr-AMC (Enzo). Fluorescence (380 nm excitation, 460 nm emission) was monitored on a microplate fluorometer (Infinite M1000, Tecan) every 1 min for 2 hours at 20° C.

### Lipidomics Sample Preparation

50,000 eggs were bleached onto the NGM plates before transferred to the RNAi plates at day 1 adults. Worms were then collected 48 hours after RNAi treatments and washed with M9 for three times. Worm pellets were snap frozen with Liquid N₂ for further processing. Total five biological repeats were collected for lipid extraction and followed previous protocol described (Benjamin et al., 2013).

### Preparation of Palmitate—BSA Conjugate

Palmitate was conjugated to BSA as described (Seahorse protocols). Briefly, sodium palmitate was solubilized in 150 mM sodium chloride by heating up to 70° C. in a water bath. Fat-free bovine serum albumin (FA-BSA) that was obtained from Sigma-Aldrich was dissolved in phosphate buffered saline (PBS) and warmed up to 37° C. Solubilized palmitate was added to BSA at 37° C. with continuous stirring. The conjugated palmitate-BSA was aliquoted and stored at −20° C. Palmitate-BSA conjugate was used to assess oxidation of exogenous fatty acid.

### Fatty Acid Oxidation Measurement of Cellular Respiration

OCR measurement was performed using the Seahorse XF96 Extracellular Flux Analyzer (Agilent Technologies). HEK 293 cells were plated in XF96 cell culture plates coated with poly-d-lysine (Sigma Aldrich) at 2×10⁴ cells/well the day prior to the experiment. Cells were equilibrated with the substrate-limited medium for 1 hour (DMEM, 0.5 mM Glucose, 1 mM Glutamax, 0.5 mM carnitine, 1% FBS, pH 7.4). Cells were then washed with the FAO medium (111 mM NaCl, 4.7 mM KCl, 1.25 mM CaCl₂, 1.2 mM Na₂HPO₄, 2 mM MgCl₂, 5 mM HEPES, 0.5 mM carnitine, 0.72M glucose, pH 7.2) and incubated in the FAO medium in a 37° C. non-CO₂ incubator for 45 min immediately before XF assay. Wells were assessed one of each treatment: BSA (5 nM), BSA (5 nM) and etomoxir (40 μM), BSA-Palmitate (20 nM), BSA-Palmitate (5 nM) and etomoxir (40 μM), all mixed with the FAO medium. All cells were probed with the XF Cell Mito Stress test (Agilent Technologies), which consists of serial treatments with oligomycin (1 μM), carbonyl cyanide 4-(trifluoromethoxy) phenylhydrazone (FCCP) (0.2 11M) and rotenone/antimycin-A (0.5 μM). These compounds were prepared in the FAO medium and were injected from the reagent ports automatically to the wells at the time indicated.

### Perhexiline Treatment

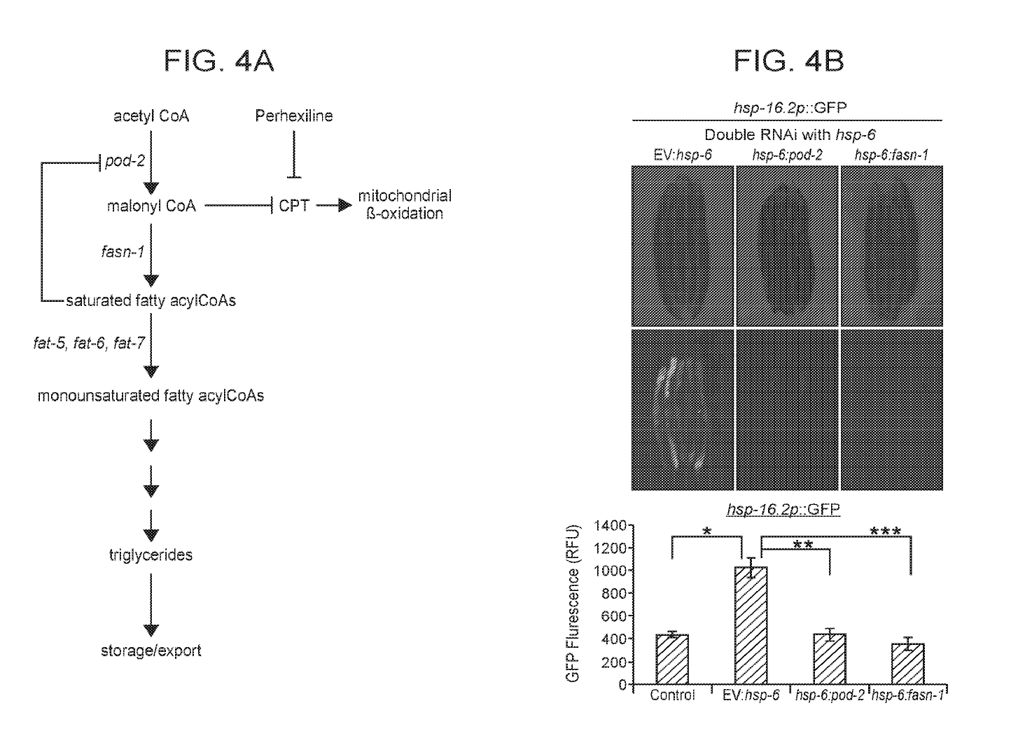

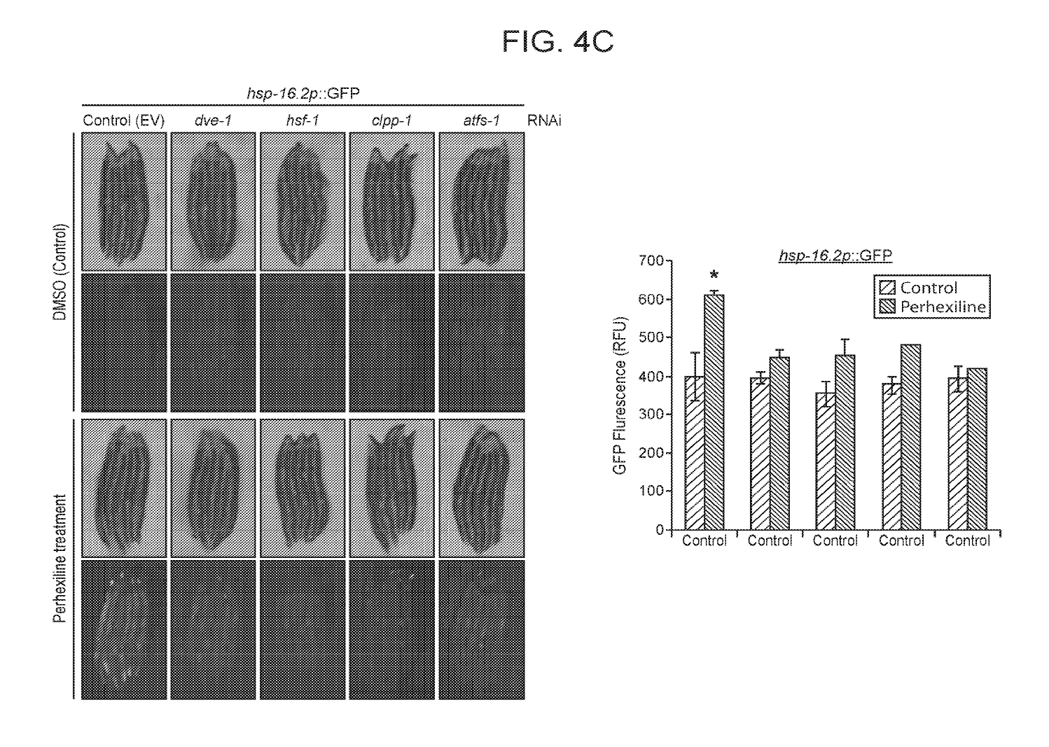

Perhexiline (Sigma) powder was dissolved in dimethylsulfoxide (DMSO) (100 mM). The stock was diluted to 1 mM and 100 μl was spotted on the E. coli OP50 plates or HT115 RNAi plates and dried before transferring the worms. Worms were transferred to new perhexiline plates every other day for three to four days until harvested for following experiment. For the perhexiline treatment with multiple numbers of RNAi (e.g., FIG. 4C), worms were transferred to the S-media containing HT115 RNAi bacteria, 100 μM of perhexiline and 1 mM of IPTG in a 96-well plate for 48 hrs.

For the cell culture experiments, cells were grown to 90% confluent and washed with PBS. Indicated concentrations of perhexiline were diluted in the cell growth media with serum and replaced in cell culture dishes. After 48 hours, cells were washed with PBS and harvested for following experiments.

### Cell Culture and Maintenance

Adult human dermal fibroblasts (Lonza) were cultured in DMEM, 10% FBS and 1× GlutaMAX (Gibco) supplemented with blasticidin (when appropriate, 2 ug/mL for selection and 1 ug/mL for maintenance of transduced cell lines). Viral particles carrying the different lengths of polyglutamine and eGFP control were made using the 3^{rd} generation system in HEK 293T cells. The viral transduction was done overnight in 8 ug/mL polybrene (Millipore). Selection was started 36 hours after the transduction for 7 days or until all the cells were eGFP positive.

### Plasmids

Exon 1 of the human huntingtin gene with different lengths of polyglutamine fused to eGFP in the C-terminal end or eGFP control were cloned into the pLenti6.3/V5-DEST vector (plasmids were a gift from Proteostasis Therapeutics).

### Oxygen Consumption Rate Measurement

Oxygen consumption of whole worms was measured using the Seahorse XF96 (Seahorse Bioscience). Worms were washed off from plates with M9 to remove residual bacteria. Then, 50 worms (10 worms/well/100 μl) were transferred to the microplate. 50 μl of M9 was added to the wells and the oxygen consumption rate was measured. All experiments were repeated at least three times.

### Electron Microscopy

Worms grown on E. coli HT115 carrying RNAi, were loaded into specimen carriers and fixed using high pressure freezing (Balzers HPM 010 High Pressure Freezer), freeze substituted in 1.0% osmium tetroxide, and 0.1% uranyl acetate in acetone at −90° C., and then slowly warmed to −10° C. and washed with pure acetone. Worms were embedded in increasing concentrations of epon resin at room temperature, transferred to flat bottom embedding capsules in pure resin, and cured at 65° C. for 48 h. Serial sections were cut at 70 nm, and placed onto formvar coated mesh copper grids. Worms were imaged using the FEI Tecnai 12 Transmission electron microscope.

### Example 1

### Cross Communication Between the UPR^{mt} and Heat Shock Response (HSR)

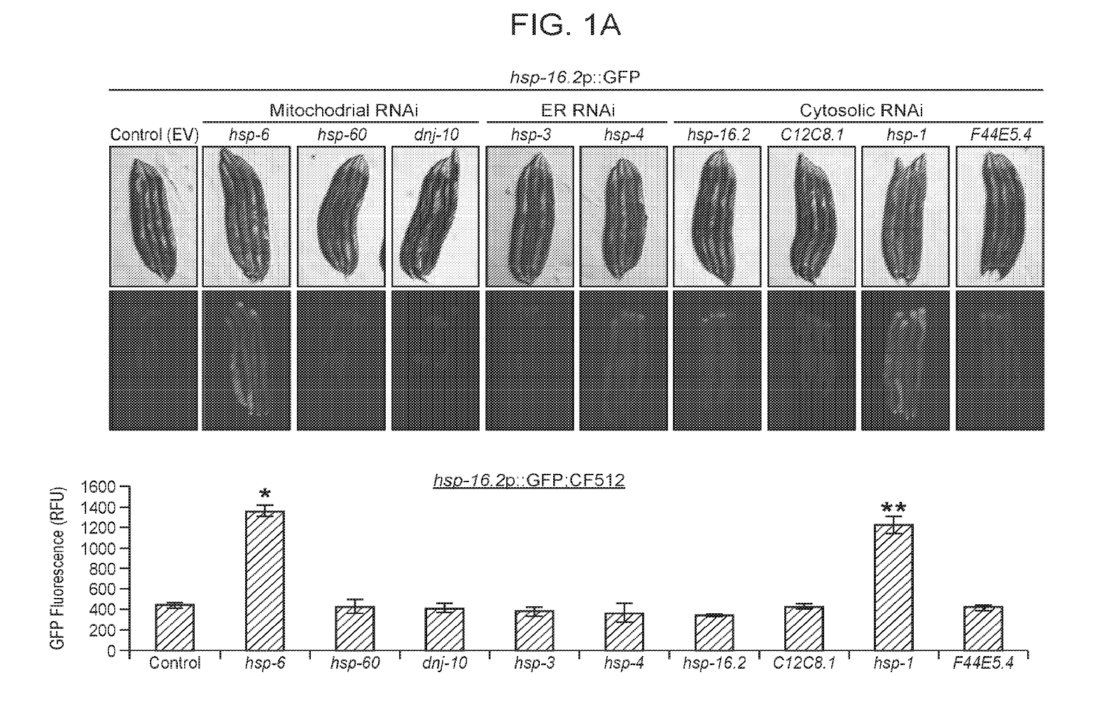

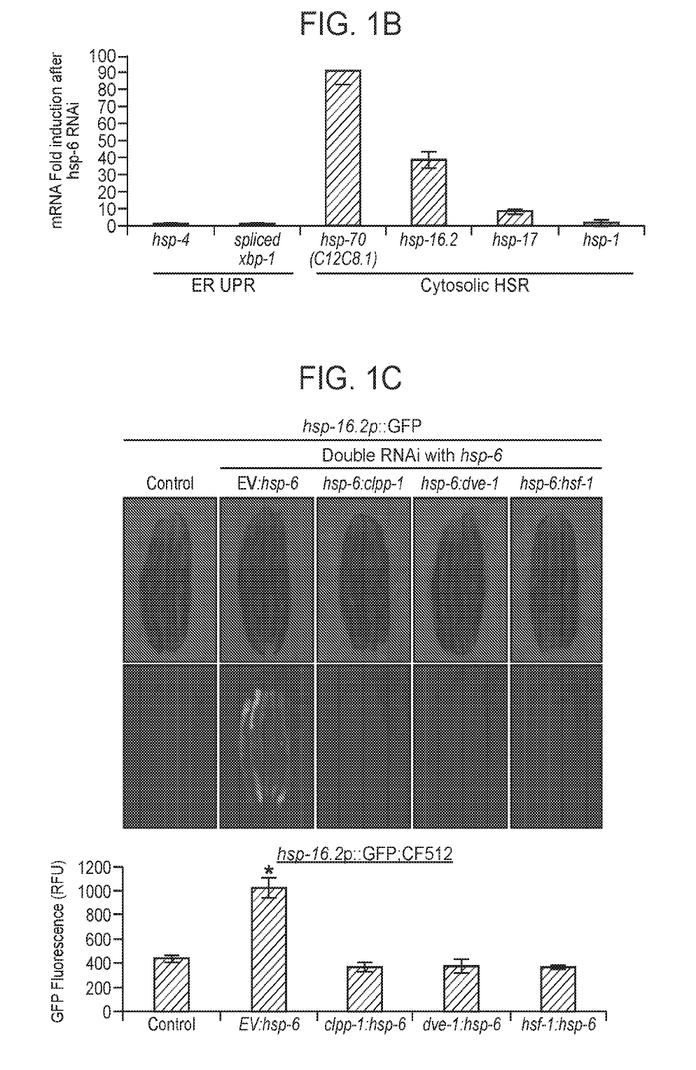

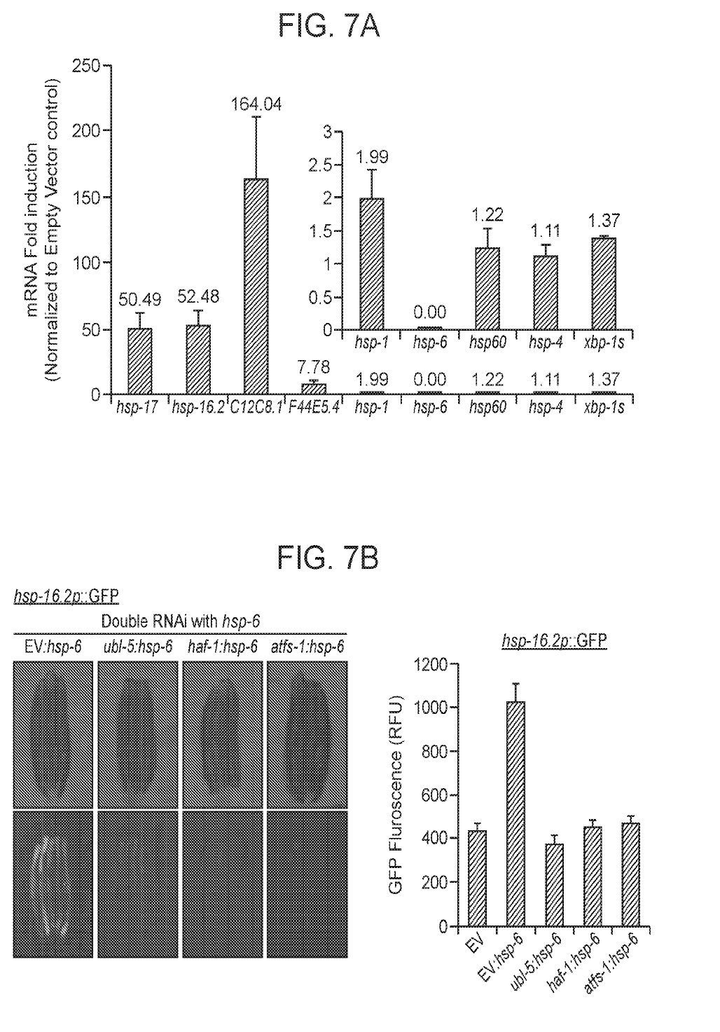

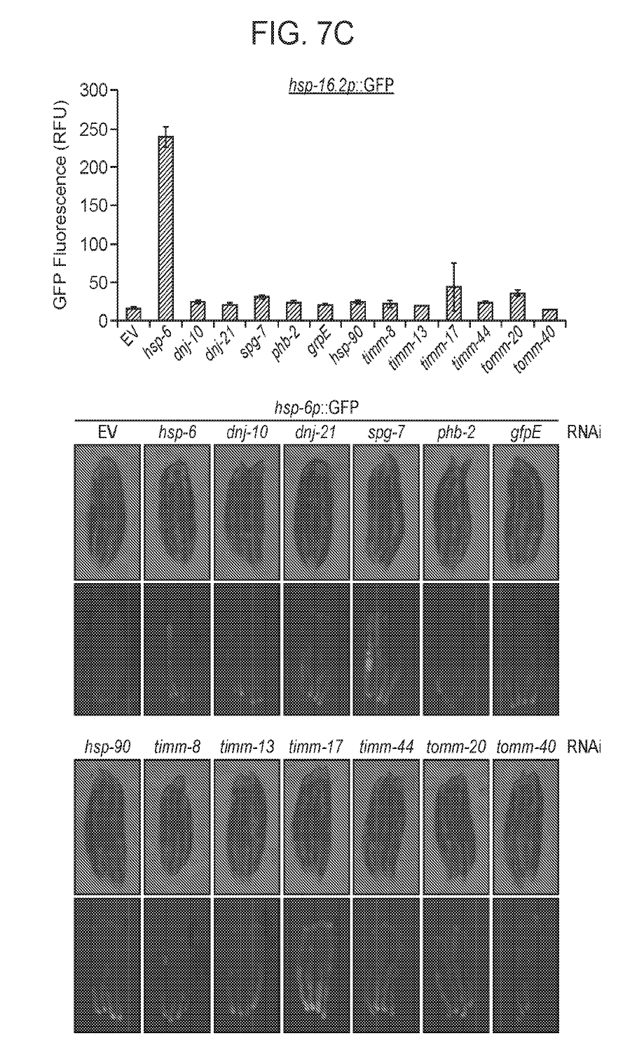

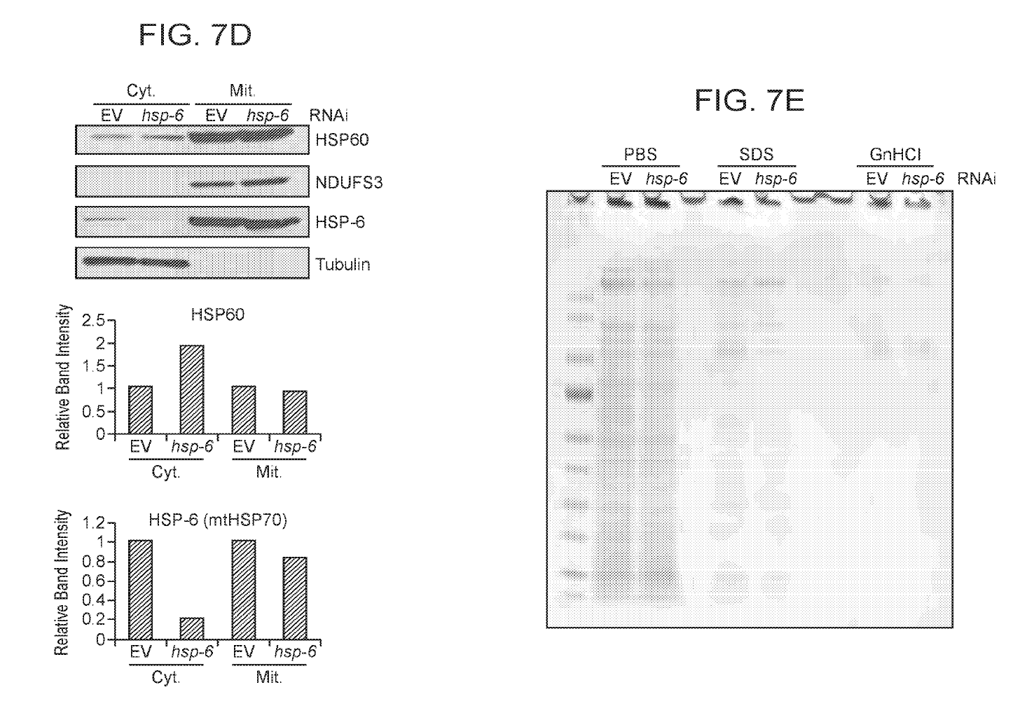

To test whether an induced unfolded protein response (UPR) in one compartment could communicate to unaffected compartment specific stress responses, the function of each Hsp70 family member was reduced using RNAi in C. elegans and its loss upon the induction of the compartment-specific UPRs was analyzed. Using this approach, RNAi against the nematode mitochondrial chaperone Hsp70, hsp-6 (Mortalin/Grp75/mtHSP70), was found to be sufficient to upregulate hsp-16.2p::GFP, a marker for the HSR, in otherwise unstressed conditions (FIG. 1A). While reduction of hsp-6 also induced the UPR^{mt}, it had no effect on the UPR^{ER} (FIG. 1A, FIG. 1B and Table 1). This effect also appeared unique: RNAi targeting any of the other eleven Hsp70 family members failed to elicit a cross-compartmental stress response. RNAi against ER resident Hsp70 family members BiP (hsp-3 and hsp-4), Hyou-1 (T14G8.3 and T24H7.2), or Stch (stc-1) had no effect on the cytosolic HSR or UPR^{mt} (FIG. 1A and Table 1). RNAi against the cytosolic hsp70 family members C12C8.1, F44E5.5, F44E5.4, hsp-110, hsp-1 or F11F1.1 likewise failed to induce the UPR^{mt} or UPR^{ER} (Table 1). hsp-6 RNAi was sufficient to upregulate not only hsp-16.2 but multiple cytosolic chaperones, suggesting a wide-spread response to the loss in mitochondrial homeostasis (FIG. 7A). For convenience, this response was named the Mitochondrial to Cytosolic Stress Response, or MCSR.

TABLE 1
Cross-compartmental UPR Induction upon Knock-down
of Individual Members of HSP70 Protein Family

| | | Mitochondria | ER | Cytosol |
|---|---|---|---|---|
| | | hsp- | hsp- | hsp- |
| | | 6p::GFP | 4p::GFP | 16.2::GFP |
| Empty Vector | | | | |
| Mitochondria | hsp-6 | + | | ++ |
| ER | stc-1 | | | |
| | T14G8.3 | | | |
| | T24H7.2 | | | |
| | hsp-3 | | + | |
| | hsp-4 | | ++ | |
| Cytosol | F44E5.4 | | | |
| | F44E5.5 | | | |
| | C12C8.1 | | | |
| | hsp-1 | | | ++ |
| | F11F1.1 | | | |
| | hsp-110 | | | |

(+, moderate induction; ++, strong induction; blank, no induction)

Because the MCSR encompassed both the mitochondrial and cytosolic stress responses, the canonical mediators of these stress pathways were tested to see if they were required for MCSR. The upregulation of the heat stress sentinel, hsp-16.2, during the MCSR was found to require hsf-1 as expected and the transcription factor dve-1, a major regulator of the UPR^{mt}, as well as the mitochondrial matrix protease clpp-1 (FIG. 1C). In addition, ubl-5, haf-1, and atfs-1 (FIG. 7B), all of which are members of the canonical UPR^{mt} were required for the MCSR.

FIG. 1A-1C. Knockdown of Mitochondrial HSP70 (hsp-6) induces cytosolic heat shock response via UPR^{mt}. (A) Mitochondrial hsp-6 and cytosolic hsp-1 RNAi induced cytosolic small heat shock protein expression. hsp-16.2p::GFP;CF512 reporter worms were tested and GFP induction was measured by COPAS biosorter (lower panel, paired T-test, mean±SD of three biological repeats). *P=0.00119, **P=0.00452. EV; empty vector control RNAi. (B) QPCR of UPR^{ER} and cytosolic HSR genes after hsp-6 RNAi (mean±SD of three biological repeats). (C) Cytosolic HSR induction after hsp-6 RNAi was dependent on both hsf-1 and UPR^{mt} components. hsp-16.2p::GFP reporter induction was measured by COPAS biosorter (lower panel, paired T-test, mean±SD of three biological repeats). *P=0.00291.

Orthologues of hsp-6, mortalin, have been associated with critical roles in the import and folding of nuclear-encoded mitochondrial matrix proteins. Therefore, induction of the cytosolic heat shock response caused by hsp-6 RNAi treatment could be due to reduced mitochondrial import, resulting in the accumulation of misfolded mitochondrial proteins in the cytosol, resulting in the eventual induction of the HSR. To test this hypothesis, the effect of reduction of mitochondrial import protein complex components on the cytosolic heat shock response was examined. Proteins targeted for the mitochondria are unfolded upon translocation across the TOM (outer mitochondrial membrane) and TIM (inner mitochondrial membrane) channels (Wiedemann et al., J. Biol. Chem. 2004, 279:14473-14476). RNAi against any of the annotated components of the import machinery was tested to see if it resulted in the MCSR. Among the components of the import machinery tested, hsp-6 RNAi was capable of inducing the HSR (FIG. 7C). Steady-state cytosolic, pre-import levels of mitochondrial proteins, including Ndufs3 and Hsp60, remained unaltered by hsp-6 RNAi treatment (FIG. 7D). Moreover, analysis of the insoluble cytosolic proteome of hsp-6 RNAi treated animals suggested that the global level of insoluble proteins in the cytosol was decreased rather than increased (FIG. 7E).

FIG. 7A-7E. hsp-6 RNAi induces a MCSR that is independent of mitochondrial import. (A) qPCR of different compartmental UPR genes after hsp-6 RNAi (mean±SD of three biological repeats). (B) UPR^{mt} components were required for induction of the cytosolic response. hsp-16.2p::GFP reporter induction was measured by COPAS biosorter (right panel, mean±SD of three biological repeats). (C) RNAi of the other mitochondrial import and quality control genes did not induce hsp-16.2p::GFP reporter (left panel), while they all moderately turned on the hsp-6p::GFP reporter (right panel, images of green fluorescent protein (GFP) induction). hsp-16.2p::GFP induction was measured via ImageExpress software after RNAi treatment (mean±SEM of three biological repeats). (D) Mitochondrial HSP60 was detected after the indicated RNAi. The western blots were quantified by normalizing to NDUFS3 expression level (mitochondrial fraction) or to Tubulin expression level (cytosolic fraction). The band intensity of each sample is compared to the respective EV control. (E) SDS-insoluble proteins after RNAi (Silver staining of SDS-PAGE gel). PBS: PBS-soluble proteins, SDS: SDS-soluble proteins from the PBS-insoluble pellet, GnHCl: GnHCl extracted proteins from the SDS-insoluble pellet.

### Example 2

### DVE-1 and HSF-1 Co-Regulate Genes Involved in Fat Metabolism

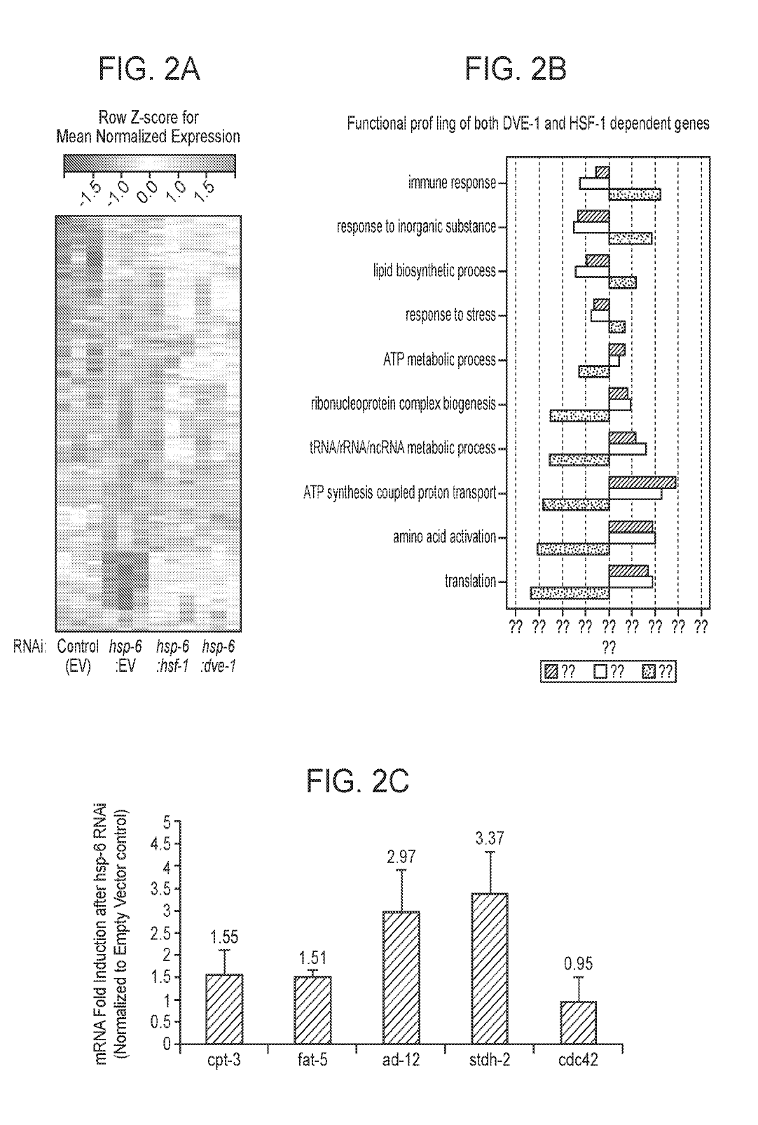

Because the induction of the MCSR due to hsp-6 RNAi required both hsf-1 and dve-1, key transcription factors required for the HSR and UPR^{mt}, respectively, which gene sets might be coordinately regulated by both factors was tested. Microarray analyses was performed and identified that the expression of 187 genes was altered by hsp-6 RNAi in comparison to control animals. 98 of 187 of these genes were coordinately regulated either by hsf-1 or dve-1 (FIG. 2A). The vast majority (66/98) of hsp-6 RNAi dependent changes in gene expression required both dve-1 and hsf-1, consistent with earlier observations of the effect of dve-1 and hsf-1 on MCSR induction (FIG. 1C). To facilitate analysis of this data in more detail, the gene expression data from the microarray analysis was analyzed for enriched Gene Ontology Biological Process (GOBP) terms with LRPath. 10 representative GOBP terms were enriched in both dve-1 and hsf-1 co-regulated genes that were altered by hsp-6 RNAi (FIG. 2B).

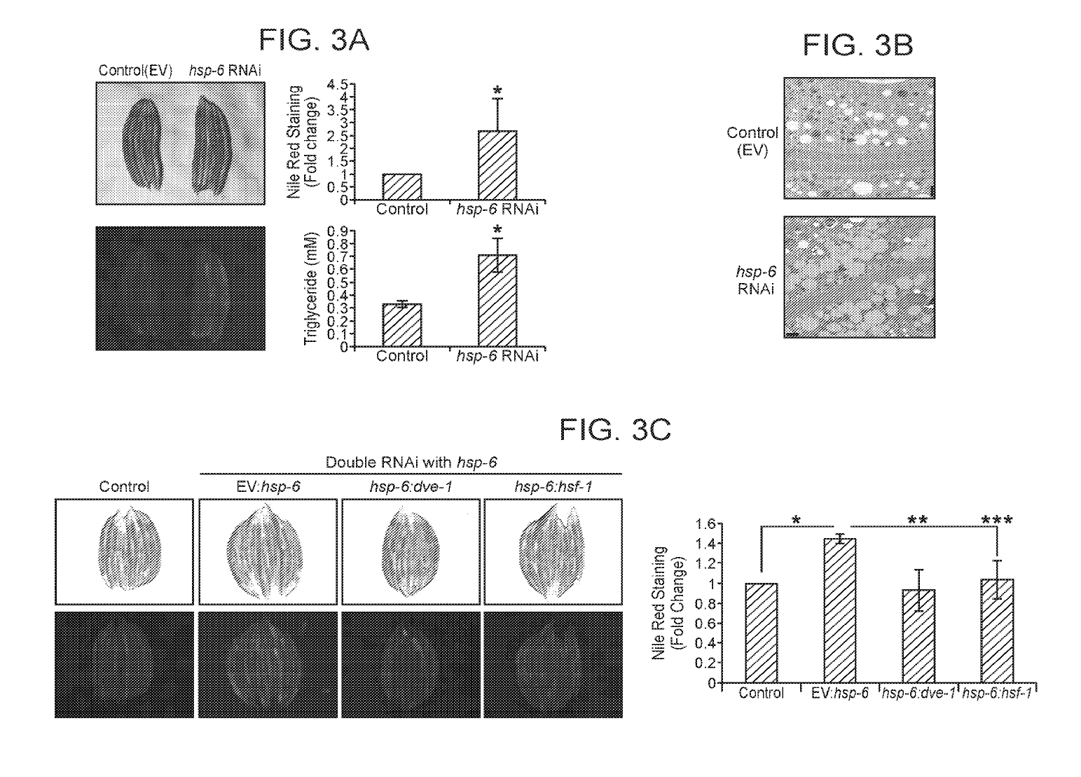

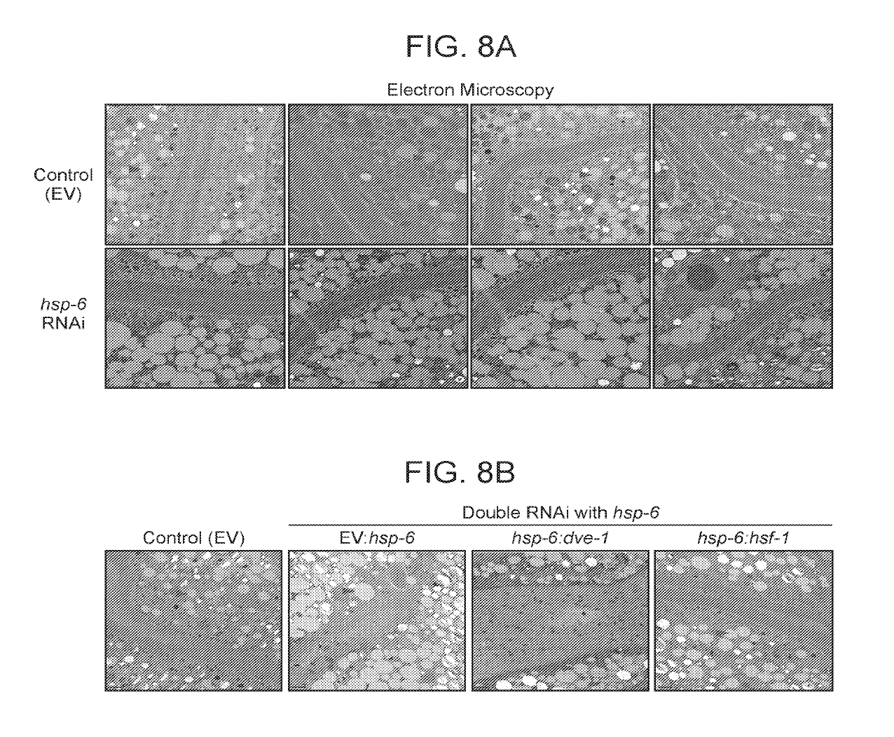

Genes involved in lipid biosynthetic process were enriched, in addition to the genes involved in the responses to different stressors (immune response, inorganic substance and endogenous stress) and the genes affecting the function of the translation machinery as expected (Lindquist, S. Dev. Biol. 1980, 77:463-479; Lindquist, S. Nature. 1981, 293:311-314; Miller et al., Proc. Natl. Acad. Sci. U.S.A 1979, 76:5222-5225). Based on the microarray data analysis, it was hypothesized that the lipid biosynthesis process may have a critical role in cross-compartmental communication between the mitochondria and cytosol. Quantitative polymerase chain reaction (QPCR) analyses of individual genes involved in lipid synthesis confirmed their upregulation in hsp-6 RNAi treated worms (FIG. 2C). Consistent with the microarray and QPCR analysis, hsp-6 RNAi treated animals also exhibited metabolic dysfunction, including the aberrant accumulation of lipid stores. hsp-6 RNAi treated animals had higher incorporation of Nile Red dye, and displayed higher triglyceride levels (FIG. 3A). Electron microscopy revealed that hsp-6 RNAi treated animals contained more lipid droplets, the primary storage organelle for fats in the intestine, which were also larger in size (FIG. 3B and FIG. 8A). The increase in lipid storage elicited by hsp-6 RNAi was also dependent upon dve-1 and hsf-1 activities (FIG. 3C and FIG. 8B).

FIG. 2A-2C. Microarray analysis suggests the DVE-1 and HSF-1 dependent gene regulation of fat metabolism. (A) Heatmap of normalized gene expression for 187 genes differentially expressed in hsp-6 RNAi relative to empty vector (EV) control. (B) DVE-1 and HSF-1 dependent GO Biological Process terms enriched in hsp-6 RNAi relative to EV. 30 GO terms were clustered to identify the 10 representative terms shown. (C) Lipid synthesis genes induction from microarray experiments was verified by QPCR after hsp-6 RNAi (mean±SD of three biological repeats).

FIG. 3A-3C. Mitochondrial HSP70, hsp-6, knockdown leads to increase in fat storage. (A) Nile Red staining on fixed worms showed increase in fat contents after hsp-6 RNAi. Nile Red staining was quantified by COPAS biosorter (paired T-test, mean±SD of three biological repeats). *P=0.03961. Triglyceride content was measured after hsp-6 RNAi (paired T-test, mean±SD of three biological repeats). *P=0.0293. (B) Electron microscopy showed increase number of lipid droplets in the intestine of hsp-6 RNAi treated worms (scale bar=2 μm, longitudinal section). Arrowheads indicate the lipid droplets. (C) Nile Red staining on fixed worms after double RNAi. Nile Red staining was quantified by COPAS biosorter (paired T-test, mean±SD of four biological repeats). *P=0.00045, **P=0.02038, ***P=0.04271

FIG. 8A-8B. (A) More electron microscopy pictures of C. elegans intestine region after hsp-6 RNAi (scale bar=2 μm, Arrowheads indicate the lipid droplets). (B) Electron microscopy pictures showed dve-1 and hsf-1 dependent lipid accumulation after hsp-6 RNAi (scale bar=2 μm, Arrowheads indicate the lipid droplets).

### Example 3

### Fat Synthesis is Required for Cytosolic Chaperone Induction

The dramatic change in lipid biosynthetic gene expression and lipid accumulation in hsp-6 RNAi treated animals suggest a concerted change in both chaperone and metabolic gene expression that is dependent upon both hsf-1 and dve-1. Thus, it was predicted that lipid disturbances might directly affect the MCSR. To test whether induction of cytosolic chaperones may be mediated by fat accumulation, expression of each of the major enzymes that are essential for fatty acid biosynthesis was reduced and evaluated for the induction of cytosolic small heat shock protein hsp-16.2 expression. pod-2 RNAi, which targets the homolog of mammalian acetyl-CoA carboxylase (accl), is predicted to decrease malonyl-CoA, a substrate for fatty acid synthesis and a regulator of fatty acid β-oxidation, trigylcerides (Mao et al., Proc. Natl. Acad. Sci. U.S.A 2006, 103:8552-8557) and lipid accumulation in adipose tissues (Mao et al., Proc. Natl. Acad. Sci. U.S.A 2009, 106:17576-17581). Reduced expression of the fatty acid synthase (fasn) fasn-1 is similarly predicted to reduce fatty acid biosynthesis resulting in the accumulation of malonyl-CoA and inhibition of fatty acid β-oxidation (Fritz et al., Oncogene. 2013, 32:5101-5110) (FIG. 4A).

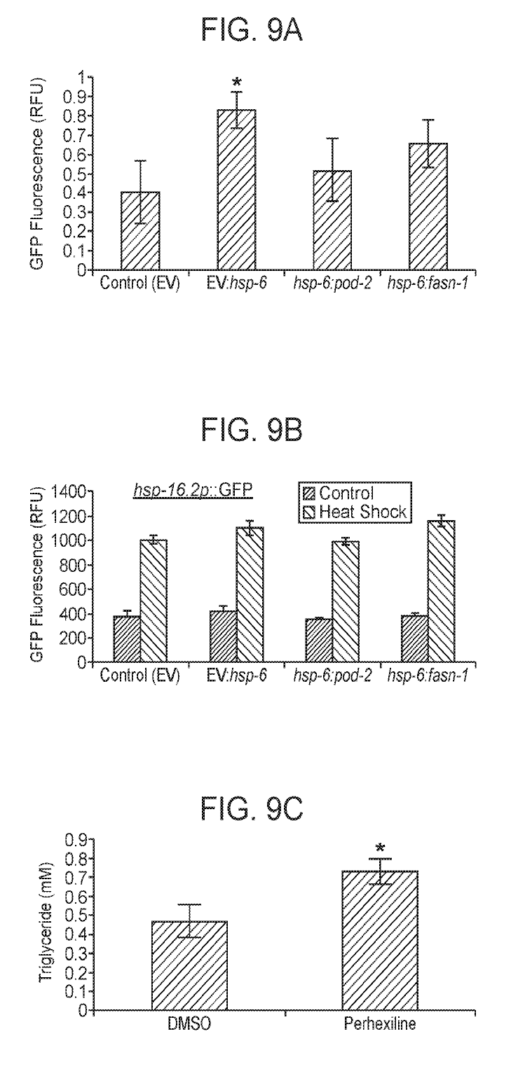

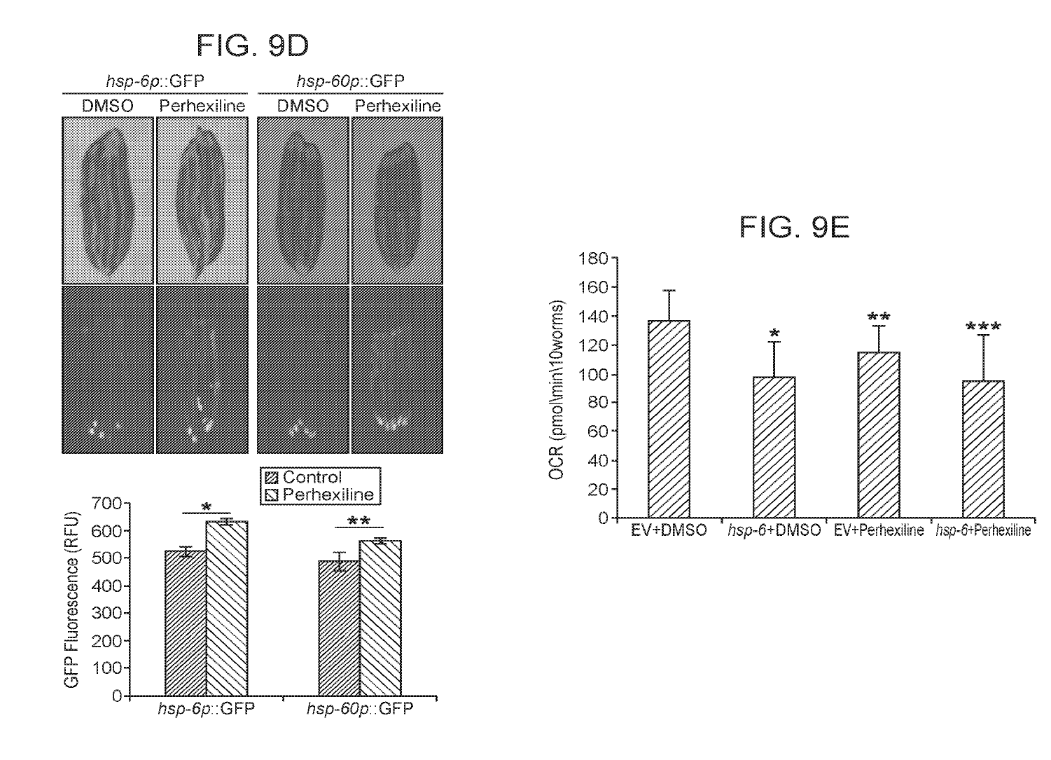

Treatment of hsp-6 RNAi treated worms with secondary RNAi against either pod-2 or fasn-1 blocked induction of cytosolic hsp-16.2 expression (FIG. 4B). Simultaneously, either pod-2 or fasn-1 RNAi blocked fat accumulation induced by hsp-6 RNAi as measured by triglyceride quantification (FIG. 9A). pod-2 and fasn-1 RNAi did not suppress hsp-16.2p::GFP expression upon heat shock treatment (FIG. 9B).

FIG. 4A-4C. Reducing fat synthesis blocks cytosolic response and inhibiting CPT activity induces cytosolic stress response. (A) Diagram showing C. elegans genes involved in fat storage pathway. pod-2; acyl-CoA carboxylase, fasn-1; fatty acid synthase, fat-5, fat-6, fat-7; delta-9 fatty acid desaturase, CPT; carnitine palmitoyltransferase. (B) Knocking down two enzymes involved in fat synthesis inhibited cytosolic response. hsp-16.2p::GFP reporter induction was measured by COPAS biosorter (lower panel, paired T-test, mean±SD of three biological repeats). *P=0.00291, **P=0.01284, ***P=0.0069. (C) CPT inhibitor perhexiline treated hsp-16.2p::GFP;CF512 reporter worms showed elevated levels of GFP that was inhibited by hsf-1 and UPR^{mt} components. GFP induction was measured by COPAS biosorter (right panel, paired T-test, mean±SD of three biological repeats). *P=0.00923.

FIG. 9A-9D. (A) Triglyceride content was measured after indicated RNAi treatment (paired T-test, mean±SD of three biological repeats). *P=0.01233. (B) Knocking down fat synthesis genes pod-2 and fasn-1 had a specific inhibitory effect on mitochondrial to cytosolic signaling while they had no effect on heat shock response. hsp-16.2p::GFP reporter induction was measured by COPAS biosorter after RNAi and heat shock (mean±SD of three biological repeats). (C) Triglyceride content was measured after perhexiline treatment (paired T-test, mean±SD of three biological repeats). *P=0.00196. (D) Perhexiline induces mitochondrial hsp-6 and hsp-60 expression. hsp-6p::GFP and reporter hsp-60p::GFP induction was measured by COPAS biosorter after perhexiline treatment (mean±SD of three biological repeats). *P=0.04626, **P=0.00397. (E) Oxygen consumption rate was measured after hsp-6 RNAi and perhexiline treatment from whole worms using Seahorse (Paired T-test, mean±SD of three biological repeats). *P=0.0104, **P=0.02007, ***P=0.01689.

### Example 4

### Ectopic Fat Accumulation Induces the HSR

Reduced fat accumulation blocks induction of the MCSR, indicating that fat accumulation is necessary for the MCSR. Fat accumulation was examined to test whether it was sufficient to induce the MCSR. Inhibition of fatty acid oxidation promotes fatty acid biosynthesis and the accumulation of lipids (Ashrafi, K. Wormbook. 2007). To block fatty acid oxidation, animals were treated with the carnitine palmitoyltransferase (CPT) inhibitor perhexiline, an inhibitor of fatty acid oxidation (FIG. 4A) (Kennedy et al., Biochem. Pharmacol. 1996, 52:273-280). Perhexiline treatment increased fat accumulation and specifically induced cytosolic hsp-16.2 expression (FIG. 4C). Perhexiline induced activation of the MCSR appears identical to hsp-6 RNAi treatment: RNAi of the UPR^{mt} components (dve-1, clpp-1 and atfs-1), as well as hsf-1, reduced the induction of the MCSR upon perhexiline treatment (FIG. 4C). Furthermore, perhexiline not only promoted fatty acid accumulation and induced the HSR (FIG. 9C) but also moderately induced UPR^{mt} (FIG. 9D). While hsp-6 RNAi reduced oxygen consumption in adults, perhexiline treatment did not further reduce overall oxygen consumption rate in these animals, suggesting that hsp-6 RNAi primarily affects mitochondrial respiration through its effects on fatty acid oxidation (FIG. 9E).

### Example 5

mtHSP70 Knockdown Improves Cytosolic Protein Homeostasis in C. elegans and Human Cells

Perturbing mitochondrial protein homeostasis by knocking down hsp-6 activates a cytosolic protein folding response and up-regulates cytosolic chaperone genes. Cytosolic protein homeostasis was tested to see if it improves when MCSR is induced. A polyglutamine proteotoxicity model in C. elegans in which YFP is fused to 35 repeats of a polygultamine (PolyQ) expansion and targeted for the cytosol of body wall muscle cells (Morley et al. Proc. Natl. Acad. Sci. U.S.A 2002, 99:10417-10422) was used. These animals exhibit an age-onset accumulation of polyQ aggregates in the muscle cells and subsequent motility defects.

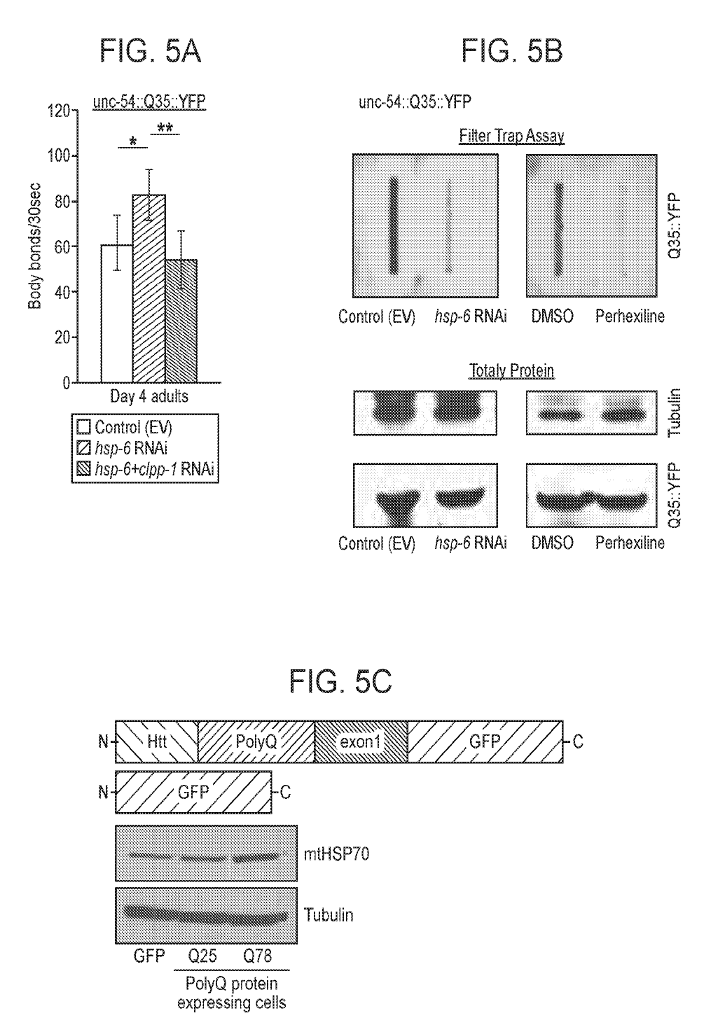

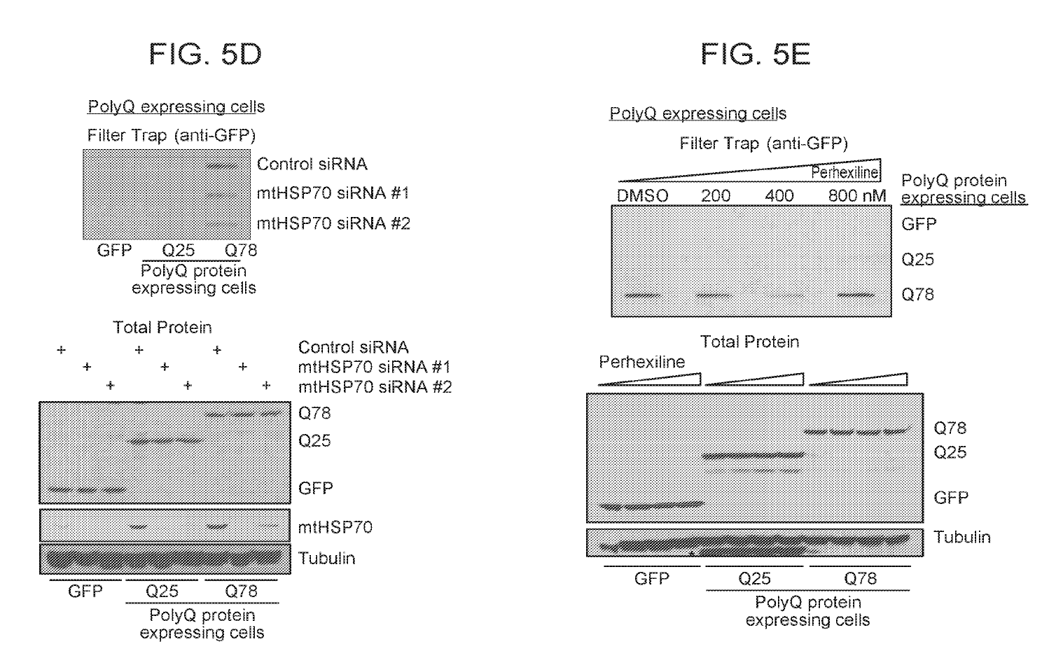

hsp-6 RNAi was found to slow the progression of motility defects in PolyQ expressing animals (FIG. 5A). clpp-1 RNAi abrogated the improved motility by hsp-6 RNAi, confirming that the improvement is through MCSR induction (FIG. 5A). Analysis of cytosolic protein aggregation, using filter trap methods, revealed that hsp-6 RNAi and perhexiline treatments resulted in decreased levels of aggregated PolyQ proteins (FIG. 5B).

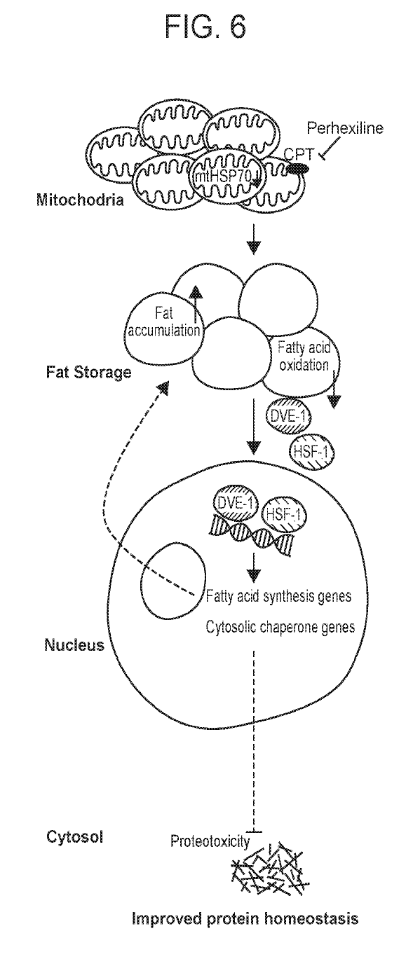

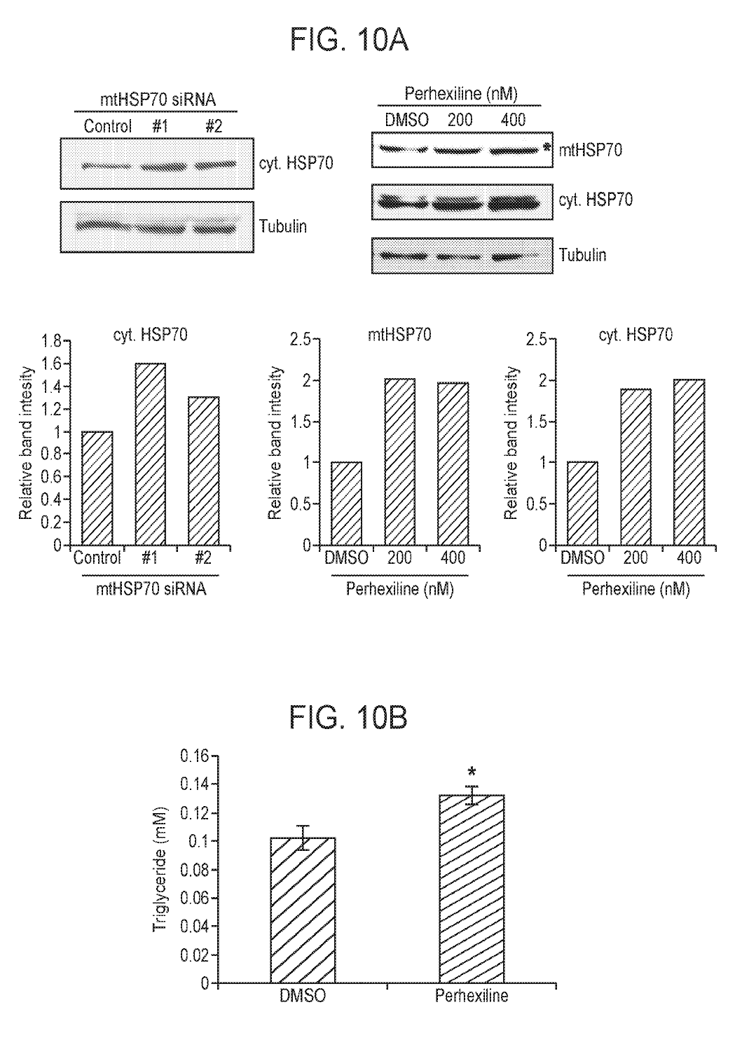

To test whether findings in C. elegans were conserved in a mammalian system, human primary fibroblast cell lines which express different lengths of polyglutamine repeats in Huntingtin exon 1 (FIG. 5C) were created. Cells with 78 polyglutamine repeats (Q78) accumulated aggregated huntingtin protein (Htt) while cells with 25 polyglutamine repeats (Q25) or GFP alone did not accumulate aggregates (FIG. 5D). mtHSP70 protein levels increased with increasing polyglutamine length (FIG. 5C) suggesting that polyQ aggregates induce the UPR^{mt}. The UPR^{mt} induced by polyQ aggregates in this cell system did not seem to alleviate cytosolic protein homeostasis, as Q78 cells still accumulated polyQ aggregates (FIG. 5D). Consistent with earlier results, proteotoxic protection was hypothesized to require the MCSR. mtHSP70 knockdown or perhexiline treatment of the human primary fibroblasts was found to induce expression of cytosolic HSP70, similar to the results from C. elegans (FIG. 10A). Furthermore, when mtHSP70 was knocked down using siRNA, a stark reduction in polyQ aggregates was observed (FIG. 5D). Similar to the results from C. elegans, perhexiline treatment also reduced polyglutamine protein aggregation in the primary human fibroblast cell lines (FIG. 5E). In addition, triglyceride levels after perhexiline treatment increased, much like in the worm (FIG. 10B). These results indicate that the MCSR is conserved from C. elegans to humans, and that mtHSP70 knockdown or CPT inhibition can send a retrograde signal to the nucleus to turn on the MCSR to improve protein homeostasis in both mitochondria and cytosol (FIG. 6).

FIG. 5A-5E. Cytosolic stress response after mitochondrial HSP70 knockdown improved cytosolic protein homeostasis in poly-Q expressing C. elegans and human primary fibroblasts. (A) AM140 worms expressing poly-Q (Q35::YFP) in body wall muscle cells were tested for motility assay after RNAi treatment. Number of body bends were measured for 30 second in M9 buffer (paired T-test, mean±SD of three biological repeats). *P=000708, **P=0.00113. (B) hsp-6 RNAi or Perhexiline treated AM140 worms showed less aggregates via filter trap assay. (C) Poly-Q expressing human primary fibroblasts were established and showed increased mtHSP expression with longer Poly-Q tract by Western blotting experiment. (D) mtHSP70 siRNA treated cells showed less aggregates via filter trap assay. Right panel shows even expression of different poly-Q length proteins, knockdown level of mtHSP70 after siRNA transfection and loading control. (E) Perhexiline treated cells also showed less aggregates on filter trap at 400 nM. Right panel shows even expression of different poly-Q length proteins and loading control. *Q25 bands from the previous probing.

FIG. 10A-10B. (A) Western blotting analysis of mtHSP70 and cytosolic HSP70 (cyt. HSP70) proteins level after siRNA treatment or perhexiline treatment on human primary fibroblasts (GFP expressing cells, *; mtHRP70 band from previous probing). Relative band intensity was analyzed using imageJ software (Bar graphs). (B) Triglyceride content of human primary fibroblasts (GFP expressing cells) was measured after perhexiline treatment (paired T-test, mean±SD of three biological repeats). *P=0.03397.

FIG. 6. Role of mtHSP70 in signaling from the mitochondria to the cytosol (MCSR) via alternating fat metabolism. mtHSP70 reduction or CPT inhibitor Perhexiline can serve as a UPR^{mt} inducer and shift the fat metabolism pathway to the fat storage pathway. Stressed mitochondria (shown in pink) would alter fatty acid metabolism towards storage. Increased fat accumulation may serve as a cytosolic signal to turn on the cytosolic response to improve cytosolic protein homeostasis. DVE-1 and HSF-1 seem to cooperate together to induce the cytosolic response upon mitochondrial perturbation.

### Example 6: Cardiolipin and Ceramide Mediate the MCSR

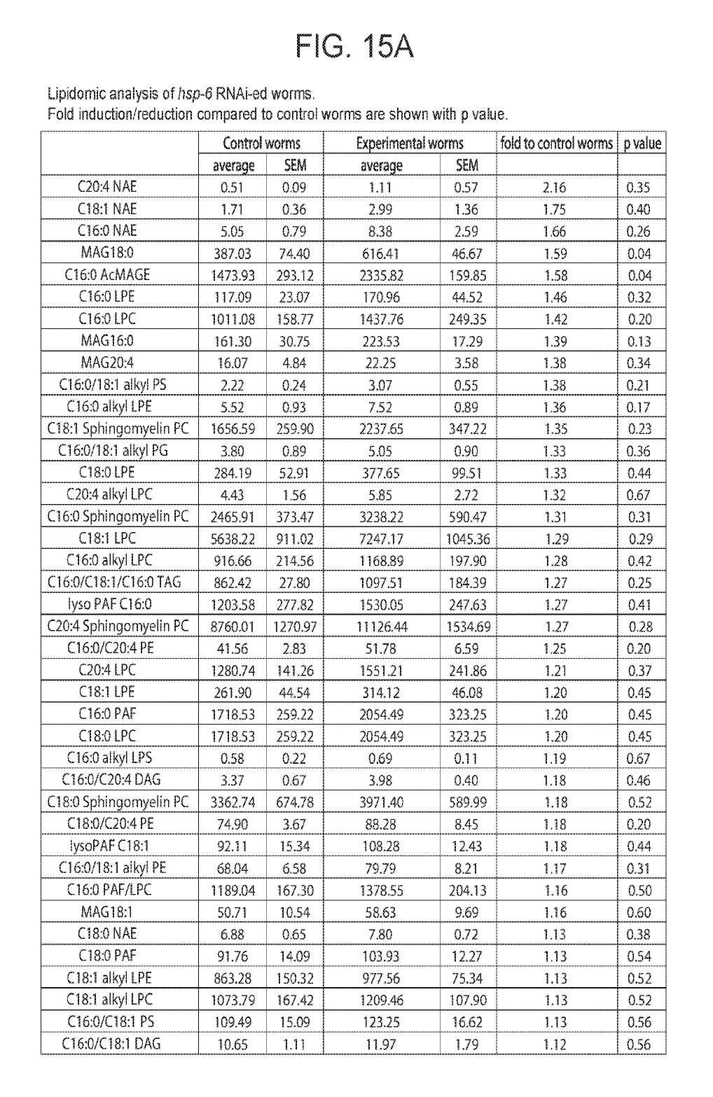

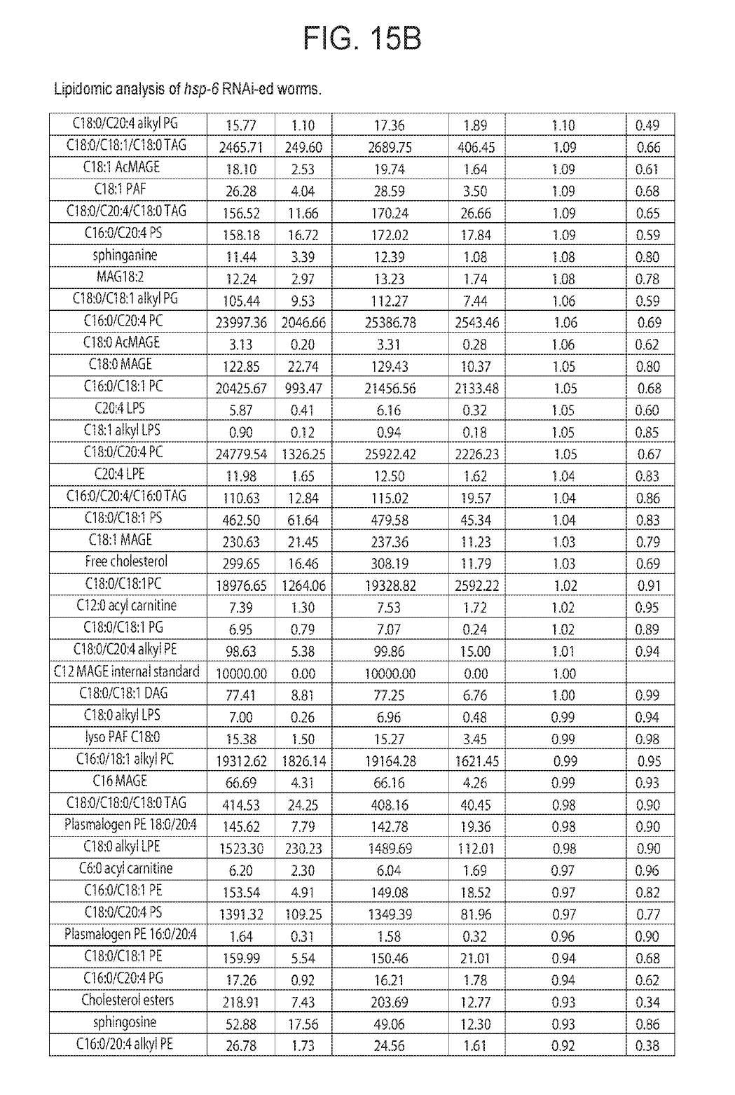

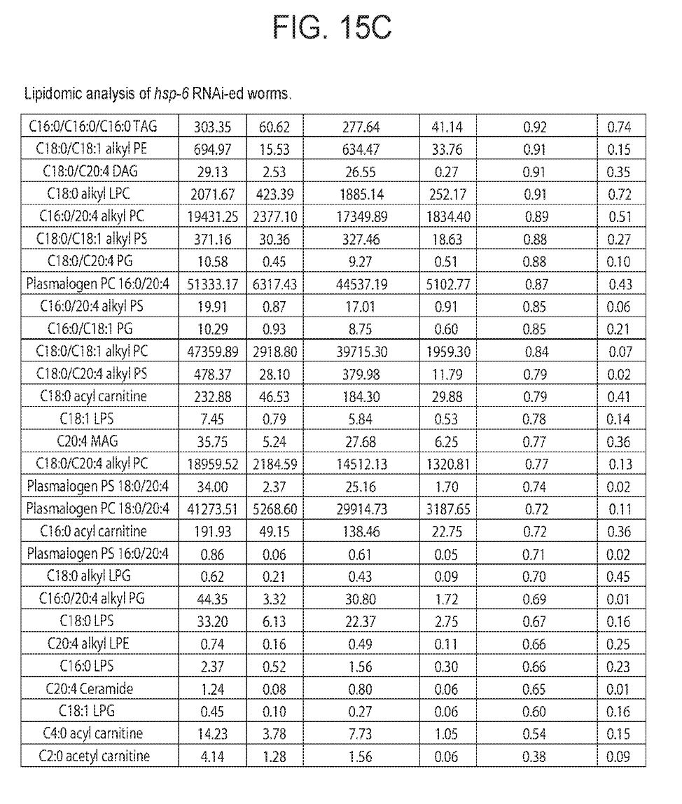

To identify the types of lipids that accumulate upon mtHSP70 knockdown, lipidomic analyses was carried out on hsp-6 RNAi treated animals. In this experiment, it was found that hsp-6 RNAi caused widespread alterations in lipid content (FIG. 15A-15C). Among these changes, levels of ether lipids, phospholipids, and precursors of phosphatidylglycerol were significantly increased (FIG. 15A-15C). Intriguingly and in stark contrast to the upregulation of these groups of lipids, ceramide levels were decreased (FIG. 15A-15C).

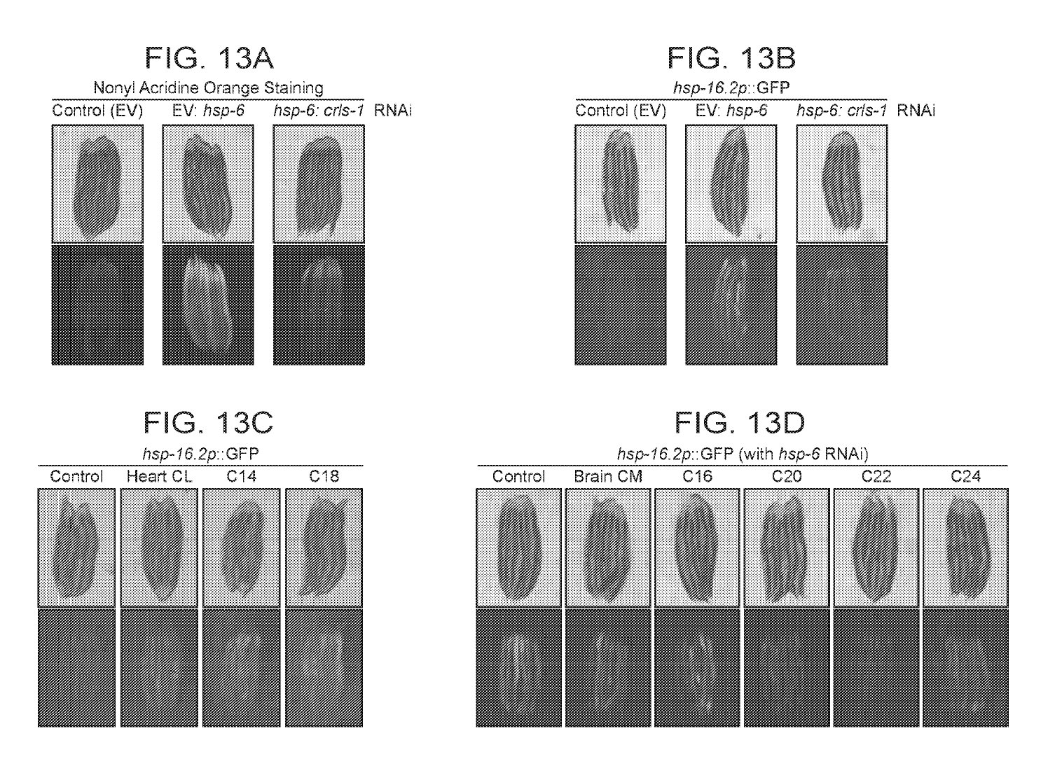

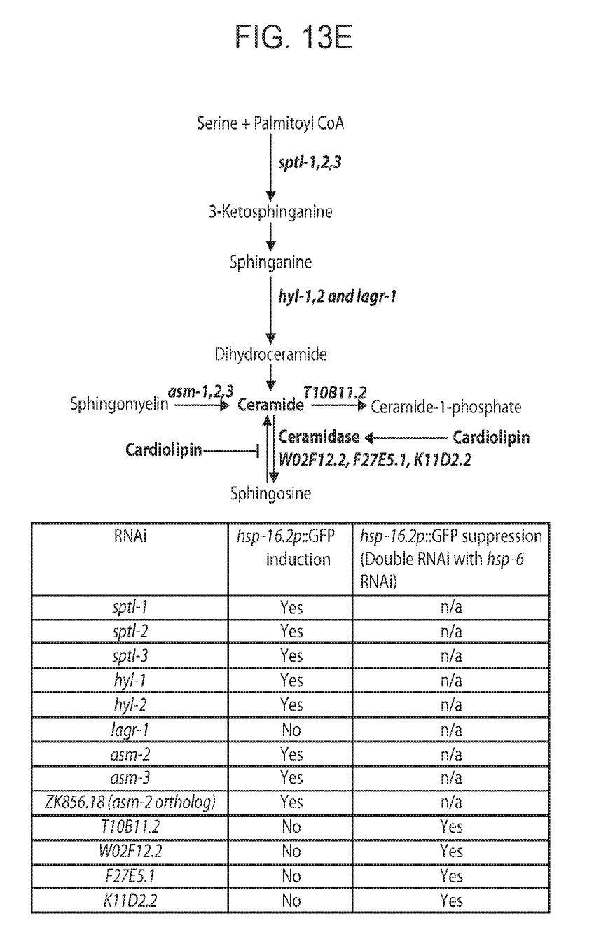

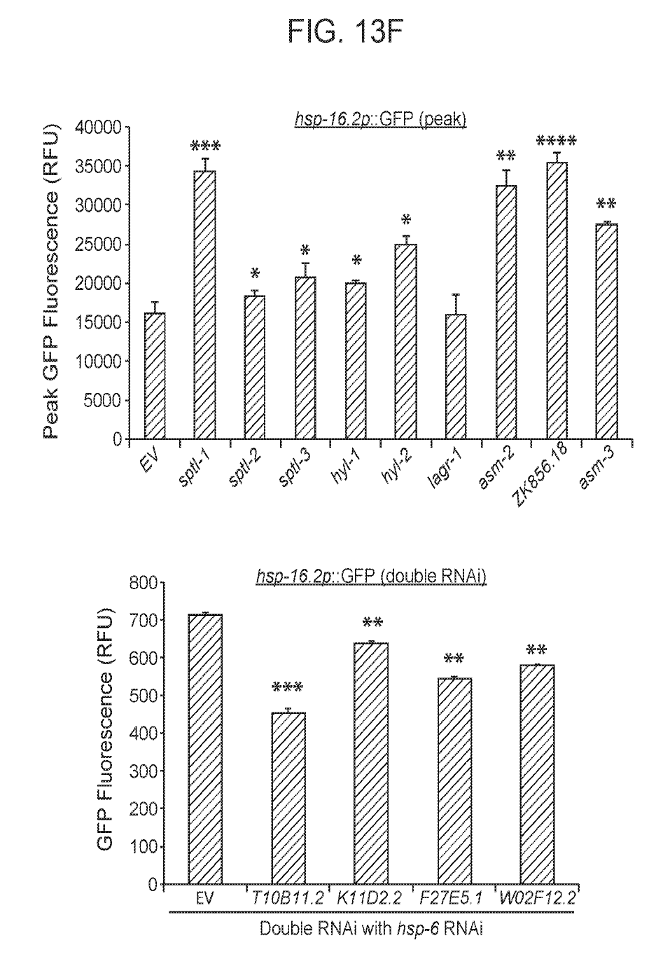

Upregulation of expression in acl-12, an ortholog of human 1pgat1 (lysophosphatidylglycerol acyltransferase 1), upon hsp-6 RNAi treatment was observed (FIG. 2C). In humans, LPGAT1 functions to convert lysophophatidic acid to phosphatidylglycerol, a precursor of cardiolipin (Yang et al., 2004). Cardiolipin is a mitochondrial phospholipid involved in mitochondrial dynamics, cristae organization, mitochondrial protein biogenesis, and respiratory supercomplex assembly and function, apoptosis, and mitophagy (Lu and Claypool, 2015; Front. Genet. 6, 3). Importantly, cardiolipins, whose lipid profiles would be grouped within many of the species seen upregulated in the lipidomic analysis, are also inhibitors of ceramide synthesis—which was down regulated in response to hsp-6 RNAi. It was hypothesized that a modulation in cardiolipin levels might be critical for the induction of the MCSR. Using nonyl acridine orange staining, it was found that hsp-6 RNAi indeed resulted in the accumulation of cardiolipin (FIG. 13A). Importantly, this effect was blocked by the additional treatment of animals with cardiolipin synthase (crls-1) RNAi (FIG. 13A). crls-1 RNAi was also sufficient to block induction of the MCSR in animals with decreased hsp-6 expression, suggesting that cardiolipin is necessary for MCSR induction (FIG. 13B).

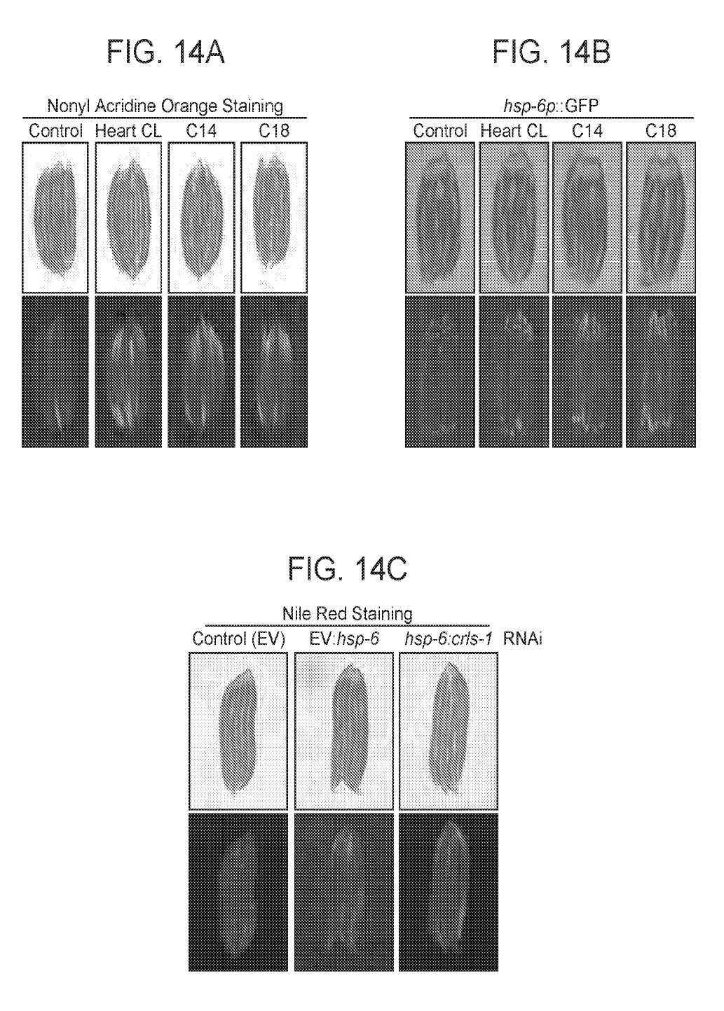

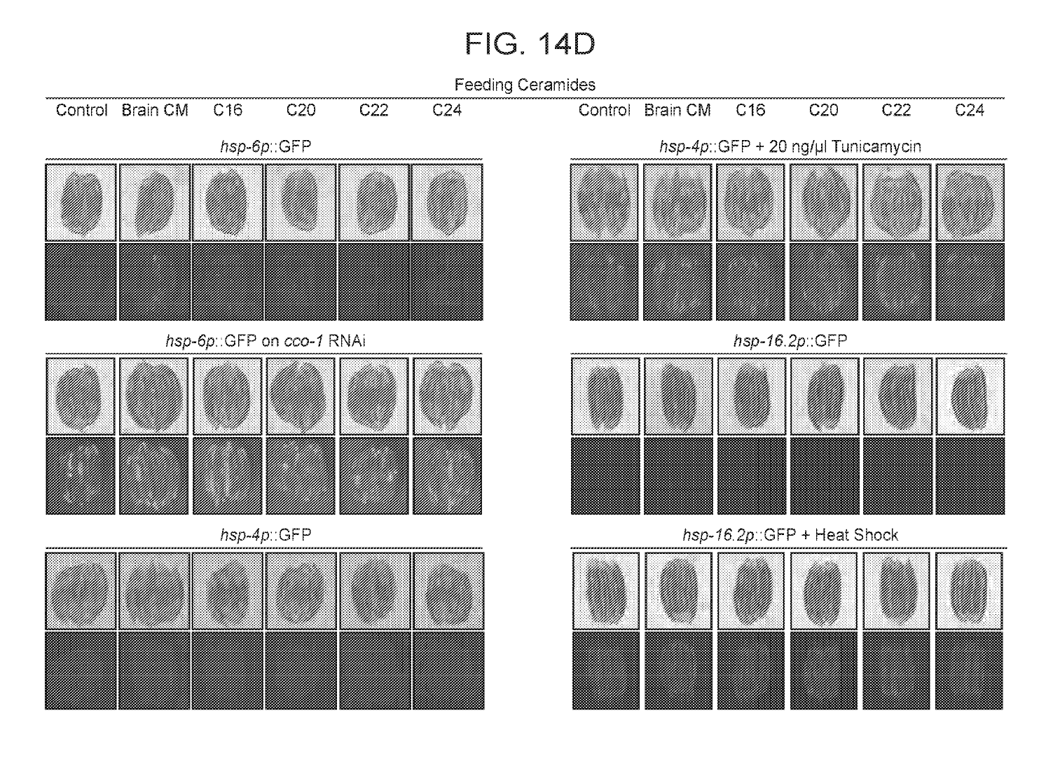

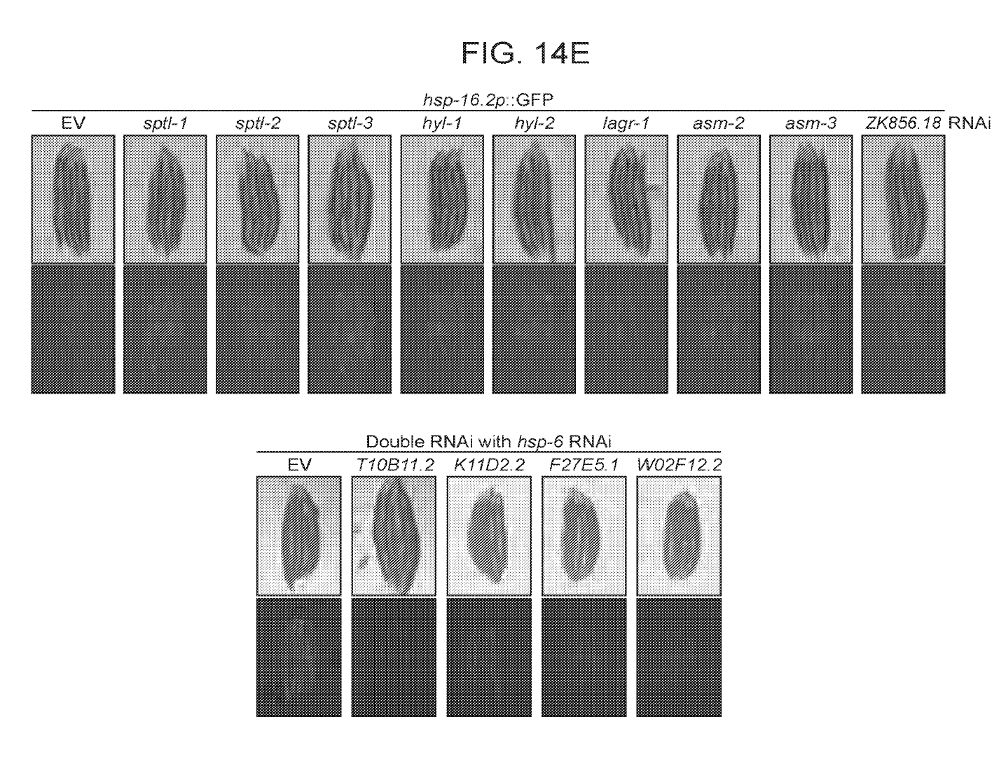

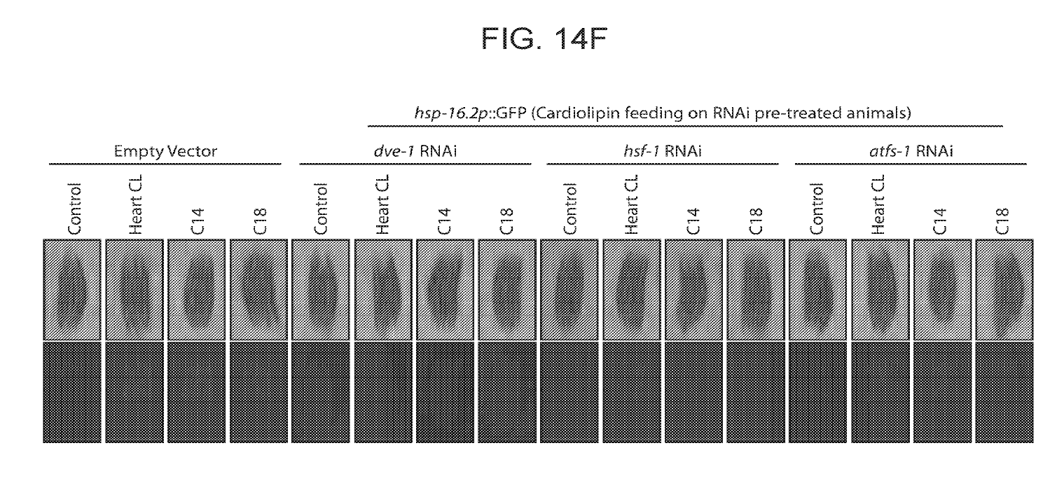

Whether exogenous cardiolipin was sufficient to drive expression of the MCSR was tested. It was found that ectopic feeding of cardiolipins to animals (FIG. 14A) was sufficient to moderately induce hsp-16.2 expression (FIG. 13C). In addition, cardiolipins were able to mildly turn on the hsp-6 reporter (FIG. 14B). In contrast, Nile Red staining remained unaffected by crls-1 RNAi (FIG. 14C). These data suggested that, while a wide range of lipids accumulates after mtHSP70 knockdown, cardiolipins play a critical role in the induction of the MCSR.

As mentioned above, cardiolipin is a potent inhibitor of the reverse activity of ceramidase (ceramide synthesis), suggesting that increased levels of cardiolipin may result in reduced ceramide levels. In view of the link between cardiolipins and reduced ceramide levels, it was hypothesized that ceramides could function to inhibit MCSR induction. Moreover, lipidomic analyses indicated reduced ceramide levels in hsp-6 knockdown worms (FIG. 15A-15C). Animals were treated with ceramides across a range of carbon chain lengths and analyzed MCSR induction. It was observed that specific ceramides were able to block the hsp-6 RNAi-induced MCSR (FIG. 13D). C20 ceramide was able to partially block the MCSR and C22 ceramide completely inhibited MCSR induction, suggesting that cardiolipin accumulation functions, in part, to affect the MCSR by inhibiting ceramide synthesis. The inhibition of the MCSR by C20 and C22 ceramide was specific, as C20 and C22 ceramide treatment did not affect UPR^{mt} caused by cco-1 knockdown (FIG. 14D). Ceramide did not induce the stress reporters tested in the absence of additional perturbations (FIG. 14D). Ceramide treatment also did not affect the compartment-specific upregulation of the UPR^{ER} by Tunicamycin treatment or of HSR by acute heat shock (FIG. 14D).

Finally, animals were treated with RNAi against each of the enzymes in the ceramide synthesis pathway (FIG. 13E). In these analyses, it was found that RNAi against enzymes involved in synthesizing ceramide were sufficient to induce the MCSR even in the absence of additional genetic perturbations (FIG. 13E, 13F and FIG. 14E). In contrast, knockdown of enzymes involved in catabolizing ceramide compromised MCSR induction upon hsp-6 RNAi (FIG. 13E, 13F and FIG. 14E). Collectively, these data suggest that ceramides are necessary and sufficient to specifically inhibit the MCSR.

FIG. 13A-13F. Cardiolipin synthesis is required for MCSR induction and inhibiting ceramide synthesis resulted in MCSR induction. (A) Nonyl Acridine Orange staining showed that hsp-6 RNAi induced cardiolipin accumulation while cardiolipin synthase (crls-1) RNAi in addition to hsp-6 RNAi blocked the cardiolipin accumulation in the wild type worms. (B) hsp-16.2p::GFP induction upon hsp-6 RNAi was inhibited by crls-1 RNAi. (C) Cardiolipin fed hsp-16.2p::GFP reporter worms showed increased GFP signal. Control; 0.5% Methanol, Heart CL; cardiolipin purified from the bovine heart, C14; C14:0 cardiolipin, C18; C18:1 cardiolipin. (D) Ceramide fed hsp-16.2p::GFP reporter worms showed inhibition of MCSR upon hsp-6 RNAi. Control; 0.5% Methanol, Brain CM; ceramide purified from the porcine brain, C16; C16 ceramide, C20; C20 ceramide, C22; C22 ceramide, C24; C24 ceramide. (E) Diagram of ceramide sysnthesis pathway. RNAi of enzymes written in red induced hsp-16.2 reporter and RNAi of enzymes written in blue reduced MCSR induction. List of enzymes that were knocked down and the RNAi result is summarized in the table. (F) Quantification of hsp-16.2p::GFP reporter induction and suppression (mean±SD of three biological repeats, * P<0.05, **P<0.01, ***P<0.001, ****P<0.0001). hsp-16.2p::GFP reporter induction in the upper panel shows the peak GFP signals from the individual worms and the hsp-16.2p::GFP reporter suppression in the lower panel shows the suppression of hsp-6 RNAi-induced MCSR (double RNAi was applied one to one ratio). See also FIG. 14A-14F and FIG. 15A-15C.

FIG. 14A-14F. Cardiolipin synthesis is required for MCSR induction and Inhibiting ceramide synthesis resulted in MCSR induction. (A) Nonyl acridine orange staining after feeding different types of cardiolipin to the worms. (B) Induction of hsp-6p::GFP after feeding the different types of cardiolipin to the reporter worms. Only mild induction in the head and hind-gut was observed. (C) Nile Red staining after double RNAi. (D) Ceramide-fed reporter worms in the presence or absence of UPR^{mt} (cco-1 RNAi), UPR^{ER} (Tunicamycin), and HSR (heat shock) induction. (E) Upper panel; hsp-16.2p::GFP reporter worms after RNAi of ceramide synthesis genes. Lower panel; hsp-16.2p::GFP reporter worms after double RNAi of ceramide catabolizing genes with hsp-6 RNAi (one to one ratio). (F) Cardiolipin-fed hsp-16.2::GFP reporter worms. Indicated RNAis were pre-treated at late L3 then transferred to the cardiolipin-containing RNAi plates at L4. The images were taken 48 hours after feeding the cardiolipin.

FIG. 15A-15C provides a table showing lipidomic analysis of hsp-6 RNAi-ed worms (hsp-6 RNAi treated worms).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of reducing protein aggregation and/or protein misfolding in a cell, the method comprising contacting the cell with an agent that modulates a mitochondrial to cytosolic stress response in the cell.

2. The method of claim 1, wherein the agent inhibits carnitine palmitoyltransferase (CPT).

3. The method of claim 2, wherein the CPT inhibitor is etomoxir, perhexiline, oxfenicine, or mildronate.

4. The method of claim 1, wherein the agent is a nucleic acid that reduces the level of mitochondrial heat shock protein 70 (mtHSP70).

5. The method of claim 4, wherein the nucleic acid is an antisense nucleic acid.

6. The method of claim 4, wherein the nucleic acid is an Shh nucleic acid or an siNA.

7. The method of any one of claims 4-6, wherein the nucleic acid comprises at least one non-phosphodiester internucleosidic linkage.

8. The method of claim 7, wherein the internucleosidic linkage is selected from phosphorothioate, phosphorodithioate, phosphoramidate, phosphorodiamidate, methylphosphonate, P-chiral linkage, chiral phosphorothioate, phosphoroselenoate, phosphorodiselenoate, phosphoroanilothioate, phosphoranilidates, phosphotriester, aminoalkylphosphotriester, alkylphosphotriester, carbonate, carbamate, morpholino carbamate, 3′-thioformacetal, morpholino, and silyl.

9. The method of any one of claims 4-8, wherein the nucleic acid comprises at least one modified nucleotide.

10. The method of claim 9, wherein the modified nucleotide is a 2′-deoxy-2′-fluoro modified nucleotide, a 2′-deoxy-modified nucleotide, a locked nucleotide, an abasic nucleotide, a 2′-ammo-modified nucleotide, a 2″-alkyl-modified nucleotide, a morpholino nucleotide, a phosphoramidate, or a non-natural base comprising nucleotide.

11. The method of claim 4, wherein at least one deoxyribose ring in the nucleic acid is substituted.

12. The antisense of claim 11, wherein at least one deoxyribose ring in the nucleic acid is substituted with a 6-membered morpholine ring.

13. The method of claim any one of claims 4-12, wherein the nucleic acid comprises at least one substituted sugar moiety.

14. The method of claim any one of claims 4-13, wherein the nucleic acid is conjugated to a lipid moiety or to poly(L-lysine).

15. A method of treating a disease or disorder associated with protein misfolding and/or aggregation in an individual, the method comprising administering to the individual an effective amount of an agent that modulates a mitochondrial to cytosolic stress response in the cell.

16. The method of claim 15, wherein the agent inhibits carnitine palmitoyltransferase (CPT).

17. The method of claim 16, wherein the CPT inhibitor is etomoxir, perhexiline, oxfenicine, or mildronate.

18. The method of claim 15, wherein the agent is a nucleic acid that reduces the level of mitochondrial heat shock protein 70 (mtHSP70).

19. The method of claim 18, wherein the nucleic acid is an antisense nucleic acid.

20. The method of claim 18, wherein the nucleic acid is an Shh nucleic acid or an siNA


SEQUENCE LISTING (2 sequences)

SEQ ID NO: 1 - protein, 840 aa, Homo sapiens
MSVVGIDLGFQSCYVAVARAGGIETIANEYSDRCTPACISFGPKNRSIGAAAKSQVISNAKNTVQGFKRFHGRAFSDPFVEAEKSNLAYDIVQLPTGLTGIKVTYMEEERNFTTEQVTAMLLSKLKETAESVLKKPVVDCVVSVPCFYTDAERRSVMDATQIAGLNCLRLMNETTAVALAYGIYKQDLPALEEKPRNVVFVDMGHSAYQVSVCAFNRGKLKVLATAFDTTLGGRKFDEVLVNHFCEEFGKKYKLDIKSKIRALLRLSQECEKLKKLMSANASDLPLSIECFMNDVDVSGTMNRGKFLEMCNDLLARVEPPLRSVLEQTKLKKEDIYAVEIVGGATRIPAVKEKISKFFGKELSTTLNADEAVTRGCALQCAILSPAFKVREFSITDVVPYPISLRWNSPAEEGSSDCEVFSKNHAAPFSKVLTFYRKEPFTLEAYYSSPQDLPYPDPAIAQFSVQKVTPQSDGSSSKVKVKVRVNVHGIFSVSSASLVEVHKSEENEEPMETDQNAKEEEKMQVDQEEPHVEEQQQQTPAENKAESEEMETSQAGSKDKKMDQPPQAKKAKVKTSTVDLPIENQLLWQIDREMLNLYIENEGKMIMQDKLEKERNDAKNAVEEYVYEMRDKLSGEYEKFVSEDDRNSFTLKLEDTENWLYEDGEDQPKQVYVDKLAELKNLGQPIKIRFQESEERPKLFEELGKQIQQYMKIISSFKNKEDQYDHLDAADMTKVEKSTNEAMEWMNNKLNLQNKQSLTMDPVVKSKEIEAKIKELTSTCSPIISKPKPKVEPPKEEQKNAEQNGPVDGQGDNPGPQAAEQGTDTAVPSDSDKKLPEMDID

SEQ ID NO: 2 - DNA, 2523 bp, Homo sapiens
atgtcggtggtgggcatagacctgggcttccagagctgctacgtcgctgtggcccgcgccggcggcatcgagactatcgctaatgagtatagcgaccgctgcacgccggcttgcatttcttttggtcctaagaatcgttcaattggagcagcagctaaaagccaggtaatttctaatgcaaagaacacagtccaaggatttaaaagattccatggccgagcattctctgatccatttgtggaggcagaaaaatctaaccttgcatatgatattgtgcagttgcctacaggattaacaggtataaaggtgacatatatggaggaagagcgaaattttaccactgagcaagtgactgccatgcttttgtccaaactgaaggagacagccgaaagtgttcttaagaagcctgtagttgactgtgttgtttcggttccttgtttctatactgatgcagaaagacgatcagtgatggatgcaacacagattgctggtcttaattgcttgcgattaatgaatgaaaccactgcagttgctcttgcatatggaatctataagcaggatcttcctgccttagaagagaaaccaagaaatgtagtttttgtagacatgggccactctgcttatcaagtttctgtatgtgcatttaatagaggaaaactgaaagttctggccactgcatttgacacgacattgggaggtagaaaatttgatgaagtgttagtaaatcacttctgtgaagaatttgggaagaaatacaagctagacattaagtccaaaatccgtgcattattacgactctctcaggagtgtgagaaactcaagaaattgatgagtgcaaatgcttcagatctccctttgagcattgaatgttttatgaatgatgttgatgtatctggaactatgaatagaggcaaatttctggagatgtgcaatgatctcttagctagagtggagccaccacttcgtagtgttttggaacaaaccaagttaaagaaagaagatatttatgcagtggagatagttggtggtgctacacgaatccctgcggtaaaagagaagatcagcaaatttttcggtaaagaacttagtacaacattaaatgctgatgaagctgtcactcgaggctgtgcattgcagtgtgccatcttatcgcctgctttcaaagtcagagaattttctatcactgatgtagtaccatatccaatatctctgagatggaattctccagctgaagaagggtcaagtgactgtgaagtcttttccaaaaatcatgctgctcctttctctaaagttcttacattttatagaaaggaacctttcactcttgaggcctactacagctctcctcaggatttgccctatccagatcctgctatagctcagttttcagttcagaaagtcactcctcagtctgatggctccagttcaaaagtgaaagtcaaagttcgagtaaatgtccatggcattttcagtgtgtccagtgcatctttagtggaggttcacaagtctgaggaaaatgaggagccaatggaaacagatcagaatgcaaaggaggaagagaagatgcaagtggaccaggaggaaccacatgttgaagagcaacagcagcagacaccagcagaaaataaggcagagtctgaagaaatggagacctctcaagctggatccaaggataaaaagatggaccaaccaccccaagccaagaaggcaaaagtgaagaccagtactgtggacctgccaatcgagaatcagctattatggcagatagacagagagatgctcaacttgtacattgaaaatgagggtaagatgatcatgcaggataaactggagaaggagcggaatgatgctaagaacgcagtggaggaatatgtgtatgaaatgagagacaagcttagtggtgaatatgagaagtttgtgagtgaagatgatcgtaacagttttactttgaaactggaagatactgaaaattggttgtatgaggatggagaagaccagccaaagcaagtttatgttgataagttggctgaattaaaaaatctaggtcaacctattaagatacgtttccaggaatctgaagaacgaccaaaattatttgaagaactagggaaacagatccaacagtatatgaaaataatcagctctttcaaaaacaaggaggaccagtatgatcatttggatgctgctgacatgacaaaggtagaaaaaagcacaaatgaagcaatggagtggatgaataacaagctaaatctgcagaacaagcagagtttgaccatggatccagttgtcaagtcaaaagagattgaagctaaaattaaggagctgacaagtacttgtagccctataatttcaaagcccaaacccaaagtggaacctccaaaagaggaacaaaaaaatgcagagcagaatggaccagtggatggacaaggagacaacccaggcccccaggctgctgagcagggtacagacacagctgtgccttcggattcagacaagaagcttcctgaaatggacattgattga